(19) United States
(12) Patent Application Publication
(10) Pub. No.: US 2016/0090383 A1
(43) Pub. Date: Mar. 31, 2016

(54) ANTIBACTERIAL PHTHALIDE DERIVATIVES

(71) Applicant: ACTELION PHARMACEUTICALS LTD., Allschwil (CH)

(72) Inventors: Christian HUBSCHWERLEN, Durmenach (FR); Verena KAEGI-EGGER, Allschwil (CH); Georg RUEEDI, Allschwil (CH); Cornelia ZUMBRUNN, Allschwil (CH)

(21) Appl. No.: 14/889,798

(22) PCT Filed: May 7, 2014
(86) PCT No.: PCT/IB2014/061262

§ 371 Date: Nov. 6, 2015

(30) Foreign Application Priority Data
May 8, 2013 (IB) PCT/IB2013/053709

(51) Int. Cl. C07D 471/06; C07D 405/12

(52) U.S. Cl. CPC C07D 471/06; C07D 405/12

(57) ABSTRACT

The invention relates to antibacterial compounds of formula I

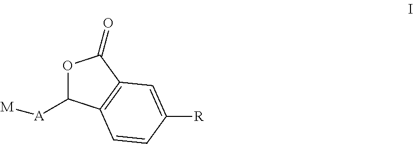

wherein R, M and A are as defined in the description, to pharmaceutical compositions containing them and uses of these compounds in the manufacture of medicaments for the treatment of bacterial infections.

The present invention concerns antibacterial phthalide derivatives, pharmaceutical compositions containing them and uses of these compounds in the manufacture of medicaments for the treatment of bacterial infections. These compounds are useful antimicrobial agents effective against a variety of human and veterinary pathogens including among others Gram-positive and Gram-negative aerobic and anaerobic bacteria and mycobacteria.

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immuno-compromised patients.

In hospital settings, an increasing number of strains of Staphylococcus aureus, Streptococcus pneumoniae, Enterococcus spp., Enterobacteriaceae and Pseudomonas aeruginosa, major sources of infections, are becoming multi-drug resistant and therefore difficult if not impossible to treat:
S. aureus is resistant to β-lactams, quinolones and now even to vancomycin;
S. pneumoniae is becoming resistant to penicillin or quinolone antibiotics and even to new macrolides;
Enteroccocci are quinolone and vancomycin resistant and β-lactam antibiotics are inefficacious against these strains;
Enterobacteriaceae are cephalosporin and quinolone resistant;
P. aeruginosa is β-lactam and quinolone resistant.

In addition, microorganisms that are causing persistent infections are increasingly being recognized as causative agents or cofactors of severe chronic diseases like peptic ulcers or heart diseases.

The diminishing number of therapeutic options for treating methicillin-resistant S. aureus (MRSA) and vancomicin-resistant enterococci (VRE) as well as their growing incidence in clinical settings make it highly desirable to find new ways of eliminating these pathogens.

WO 2009/104159 describes antibacterial compounds of formula A1 comprising a substituted oxazolidinone moiety linked to, for example, a 2-oxoquinolin-1-yl moiety:

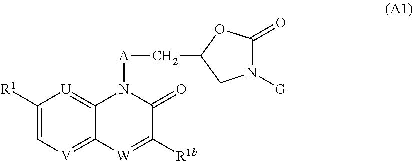

WO 2010/041194 describes antibacterial compounds of formula A2 comprising a substituted oxazolidinone moiety linked to, for example, a 4-oxo-2,4-dihydro-1H-pyrrolo[3,2,1-ij]quinolin-1-yl moiety:

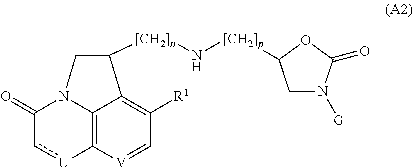

The instant invention provides novel antibacterial compounds based on a phthalide motif, namely the compounds of formula I described herein.

1) A first embodiment of the invention relates to compounds of formula I

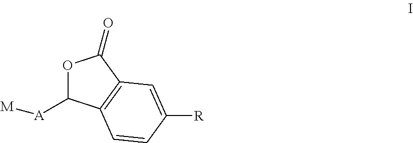

wherein

R represents phenyl optionally substituted with one or two halogen (notably fluorine); cyclopenten-1-yl or cyclohexen-1-yl; or (C₂-C₅)alkenyl; and

the groups A and M are as follows:
A represents —NH—CH₂—CH₂—CH₂—, —NH—CH₂—CH₂—NH—CO—; or —CH₂—NH—CO—CH₂—; and
M represents

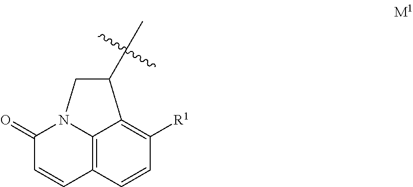

wherein R¹ represents halogen (especially R¹ represents fluorine);
or A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and
M represents

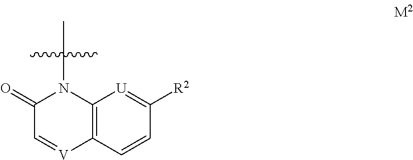

wherein R² represents (C₁-C₃)alkoxy (especially methoxy) or halogen (especially fluorine); U represents CH or N; and V represents CH or N.

Definitions provided herein are intended to apply uniformly to the compounds of formula I as defined in any one of embodiments 1) to 13), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The term “alkyl”, used alone or in combination, refers to a saturated straight or branched chain hydrocarbon group containing one to six carbon atoms. The term “(Cₓ-C_{y})alkyl” (x and y each being an integer), refers to an alkyl group as defined before, containing x to y carbon atoms. For example a (C₁-C₄)alkyl group contains from one to four carbon atoms. Examples of alkyl groups are methyl, ethyl, propyl, isopropyl, n-butyl, isobutyl, sec.-butyl and tert.-butyl. Preferred are methyl and ethyl. Most preferred is methyl.

The term “alkoxy”, used alone or in combination, refers to an alkyl-O— group wherein the alkyl group is as defined before. The term “(Cₓ-C_{y})alkoxy” (x and y each being an integer) refers to an alkoxy group as defined before containing x to y carbon atoms. For example a (C₁-C₃)alkoxy group means a group of the formula (C₁-C₃)alkyl-O— in which the term “(C₁-C₃)alkyl” has the previously given significance. Examples of alkoxy groups are methoxy, ethoxy, n-propoxy, and isopropoxy. Preferred is methoxy.

The term “alkenyl”, used alone or in combination, refers to a straight or branched hydrocarbon chain containing two to five carbon atoms and one carbon-carbon double bond. The term “(Cₓ-C_{y})alkenyl” (x and y each being an integer), refers to an alkenyl group as defined before containing x to y carbon atoms. For example a (C₂-C₅)alkenyl group contains from two to five carbon atoms. Preferably such alkenyl groups are attached to the rest of the molecule at a carbon atom bearing the double bond. Examples of alkenyl groups vinyl, prop-1-en-1-yl, 2-methylprop-1-en-1-yl, but-2-en-2-yl, and allyl. Preferred are 2-methylprop-1-en-1-yl and prop-1-en-1-yl. Most preferred is 2-methylprop-1-en-1-yl.

The term “halogen” refers to fluorine, chlorine, bromine or iodine. Preferred are fluorine and chlorine. For the substituents R¹, R², and for optional substituents of R being a phenyl group the term preferably refers to fluorine.

Particular examples of R representing optionally substituted phenyl groups are phenyl, 2,3-difluoro-phenyl, 2,4-difluoro-phenyl, 2,5-difluoro-phenyl, 3,4-difluoro-phenyl, 3,5-difluoro-phenyl, 2-fluoro-phenyl, and 3-fluoro-phenyl. Preferred are phenyl, 2,3-difluoro-phenyl, 2,5-difluoro-phenyl, 3,5-difluoro-phenyl, 2-fluoro-phenyl, and 3-fluoro-phenyl.

The term “quinolone-resistant”, “methicillin-resistant” or “vancomycin-resistant” associated to a bacterial strain, when used in this text, refer to a bacterial strain against which respectively ciprofloxacin, methicillin or vancomycin have a Minimal Inhibitory Concentration of at least 16 mg/1 (said Minimal Inhibitory Concentration being measured with the standard method described in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA, 2006). The term “multi-drug-resistant” associated to a bacterial strain when used in this text, refers to a bacterial strain that is resistant to more than three classes of antibiotics.

In this patent application, a bond interrupted by a wavy line shows the point of attachment of the radical drawn. For example, the radical drawn below

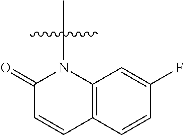 [SMILES: *n1c(=O)ccc2ccc(F)cc21]

is the 7-fluoro-2-oxoquinolin-1-yl group. In case of bivalent radicals bearing two points of attachment as for example the groups A, such radicals are to be read from left to right. For example A representing the bivalent radical —NH—CH₂—CH₂—CH₂—, means that the nitrogen atom is attached to the group M, whereas the terminal methylene group is attached to the phthalide ring.

The compounds of formula I contain at least one stereogenic center in position 1 of the 3-oxo-1,3-dihydro-isobenzofurane moiety. It is understood that both absolute configurations of said chiral center are comprised in the scope of the present invention. In case M represents M¹, the compounds of formula I contain at least one further stereogenic center in position 1 of the 4-oxo-2,4-dihydro-1H-pyrrolo[3,2,1-ij]quinolin-1-yl moiety. It is understood that both absolute configurations as depicted in radicals M¹⁻¹ and M¹⁻² below are comprised in the scope of the present invention. In addition, the compounds of formula I may contain further stereogenic or asymmetric centers, such as one or more asymmetric carbon atoms. The compounds of formula I may thus be present as mixtures of stereoisomers or preferably as pure stereoisomers. Mixtures of stereoisomers may be separated in a manner known to a person skilled in the art.

Various embodiments of the invention are presented hereafter:

2) A second embodiment relates to the compounds of formula I according to embodiment 1), wherein:

R represents phenyl; phenyl substituted with one or two fluorine substituents; cyclopenten-1-yl; cyclohexen-1-yl; 2-methylprop-1-en-1-yl; or prop-1-en-1-yl; and

the groups A and M are as follows:
A represents —NH—CH₂—CH₂—CH₂—, —NH—CH₂—CH₂—NH—CO—; or —CH₂—NH—CO—CH₂—; and
M represents

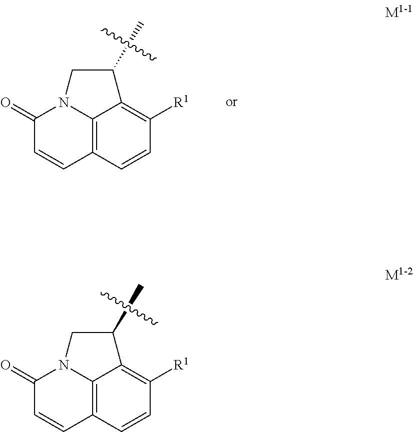

wherein R¹ independently represents halogen (especially R¹ represents fluorine);
or A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and
M represents

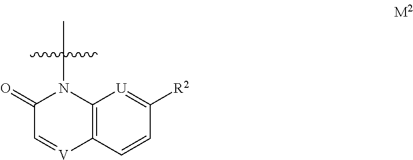

wherein R² represents (C₁-C₃)alkoxy (especially methoxy) or halogen (especially fluorine); U represents CH or N; and V represents CH or N.

3) A third embodiment relates to the compounds of formula I according to embodiment 1), wherein:

R represents phenyl; phenyl substituted with one or two fluorine substituents; cyclopenten-1-yl; cyclohexen-1-yl; 2-methylprop-1-en-1-yl; or prop-1-en-1-yl; and

the groups A and M are as follows:
A represents —NH—CH₂—CH₂—CH₂—, —NH—CH₂—CH₂—NH—CO—; or —CH₂—NH—CO—CH₂—; and
M represents

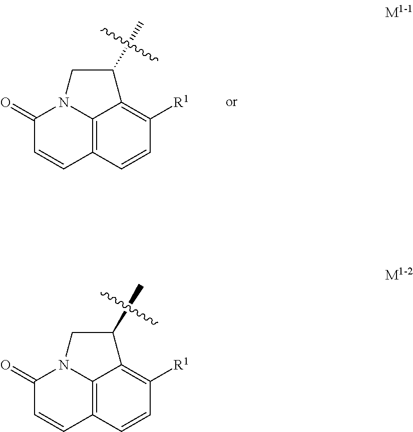

wherein R¹ represents fluorine;
or A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and
M represents

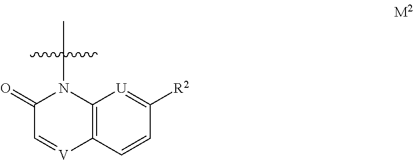

wherein
   R² represents methoxy; U represents N; and V represents N; or
   R² represents fluorine; U represents CH; and V represents CH.

4) A further embodiment relates to the compounds according to embodiment 1), wherein:

A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and

M represents

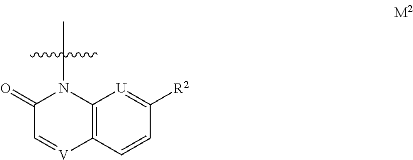

wherein R² represents (C₁-C₃)alkoxy (especially methoxy) or halogen (especially fluorine); U represents CH or N; and V represents CH or N.

5) A further embodiment relates to the compounds according to embodiment 1), wherein:

A represents —NH—CH₂—CH₂—CH₂—, —NH—CH₂—CH₂—NH—CO—; or —CH₂—NH—CO—CH₂—; and

M represents

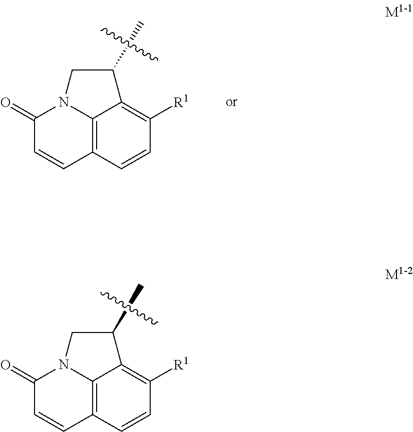

wherein R¹ independently represents halogen (especially R¹ represents fluorine).

6) A further embodiment relates to the compounds according to any one of embodiments 1) to 4), wherein, in case M represents M²,
R² represents methoxy; U represents N; and V represents N; or
R² represents fluorine; U represents CH; and V represents CH.

7) A further embodiment relates to the compounds according to any one of embodiments 1) to 4), wherein, in case M represents M²,
A represents —CH₂—CH₂—NH—CH₂—CH₂—; and R² represents methoxy; U represents N; and V represents N; or
A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and R² represents fluorine; U represents CH; and V represents CH.

8) A further embodiment relates to the compounds according to any one of embodiments 1) to 7), wherein R represents
phenyl or phenyl substituted with one or two fluorine;
cyclopenten-1-yl or cyclohexen-1-yl; or
2-methylprop-1-en-1-yl or prop-1-en-1-yl;

wherein preferably R represents phenyl, 2,5-difluorophenyl, 3,5-difluorophenyl, 2-methylprop-1-en-1-yl, or cyclohexen-1-yl.

9) A further embodiment relates to the compounds according to any one of embodiments 1) to 8), wherein the compounds are present in enantiomerically enriched form, wherein the 3-oxo-1,3-dihydro-isobenzofurane moiety has the absolute configuration as depicted in formula Ia:

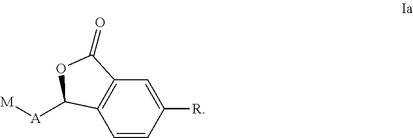

10) A further embodiment relates to the compounds according to any one of embodiments 1) to 8), wherein the compounds are present in enantiomerically enriched form, wherein the 3-oxo-1,3-dihydro-isobenzofurane moiety has the absolute configuration as depicted in formula Ib:

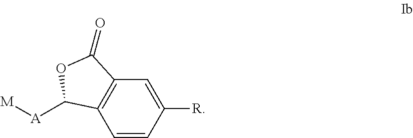

11) Particular compounds of formula I according to embodiment 1) are selected from the group consisting of:
N—((R)-9-Fluoro-4-oxo-1,2-dihydro-4H-pyrrolo[3,2,1-ij]quinolin-1-ylmethyl)-2-(3-oxo-5-phenyl-1,3-dihydro-isobenzofuran-1-yl)-acetamide;
6-Methoxy-4-{2-[2-(3-oxo-5-phenyl-1,3-dihydro-isobenzofuran-1-yl)-ethylamino]-ethyl}-4H-pyrido[2,3-b]pyrazin-3-one;
7-Fluoro-1-{2-[2-(3-oxo-5-phenyl-1,3-dihydro-isobenzofuran-1-yl)-ethylamino]-ethyl}-1H-quinolin-2-one;
N-[2-(7-Fluoro-2-oxo-2H-quinolin-1-yl)-ethyl]-2-(3-oxo-5-phenyl-1,3-dihydro-isobenzofuran-1-yl)-acetamide;
N-[2-(7-Fluoro-2-oxo-2H-quinolin-1-yl)-ethyl]-2-[5-(3-fluoro-phenyl)-3-oxo-1,3-dihydro-isobenzofuran-1-yl]-acetamide;
N-[2-(7-Fluoro-2-oxo-2H-quinolin-1-yl)-ethyl]-2-[5-(2-fluoro-phenyl)-3-oxo-1,3-dihydro-isobenzofuran-1-yl]-acetamide;
(S)-9-Fluoro-1-[3-(3-oxo-5-phenyl-1,3-dihydro-isobenzofuran-1-yl)-propylamino]-1,2-dihydro-pyrrolo[3,2,1-ij]quinolin-4-one;
2-[5-(2,3-Difluoro-phenyl)-3-oxo-1,3-dihydro-isobenzofuran-1-yl]-N-[2-(7-fluoro-2-oxo-2H-quinolin-1-yl)-ethyl]-acetamide;
2-[5-(2,5-Difluoro-phenyl)-3-oxo-1,3-dihydro-isobenzofuran-1-yl]-N-[2-(7-fluoro-2-oxo-2H-quinolin-1-yl)-ethyl]-acetamide;
2-[5-(3,4-Difluoro-phenyl)-3-oxo-1,3-dihydro-isobenzofuran-1-yl]-N-[2-(7-fluoro-2-oxo-2H-quinolin-1-yl)-ethyl]-acetamide;
2-[5-(3,5-Difluoro-phenyl)-3-oxo-1,3-dihydro-isobenzofuran-1-yl]-N-[2-(7-fluoro-2-oxo-2H-quinolin-1-yl)-ethyl]-acetamide;
2-(5-Cyclopent-1-enyl-3-oxo-1,3-dihydro-isobenzofuran-1-yl)-N-[2-(7-fluoro-2-oxo-2H-quinolin-1-yl)-ethyl]-acetamide;
3-Oxo-5-phenyl-1,3-dihydro-isobenzofuran-1-carboxylic acid [3-(7-fluoro-2-oxo-2H-quinolin-1-yl)-propyl]-amide;
3-Oxo-5-phenyl-1,3-dihydro-isobenzofuran-1-carboxylic acid [2-((R)-9-fluoro-4-oxo-1,2-dihydro-4H-pyrrolo[3,2,1-ij]quinolin-1-ylamino)-ethyl]-amide;
1-(2-{2-[5-(2,4-Difluoro-phenyl)-3-oxo-1,3-dihydro-isobenzofuran-1-yl]-ethylamino}-ethyl)-7-fluoro-1H-quinolin-2-one;
2-(5-Cyclohex-1-enyl-3-oxo-1,3-dihydro-isobenzofuran-1-yl)-N-[2-(7-fluoro-2-oxo-2H-quinolin-1-yl)-ethyl]-acetamide;
N-[2-(7-Fluoro-2-oxo-2H-quinolin-1-yl)-ethyl]-2-[5-(2-methyl-propenyl)-3-oxo-1,3-dihydro-isobenzofuran-1-yl]-acetamide;
2-(7-Fluoro-2-oxo-2H-quinolin-1-yl)-N-[2-(3-oxo-5-phenyl-1,3-dihydro-isobenzofuran-1-yl)-ethyl]-acetamide;
N-{2-[5-(2,5-Difluoro-phenyl)-3-oxo-1,3-dihydro-isobenzofuran-1-yl]-ethyl}-2-(7-fluoro-2-oxo-2H-quinolin-1-yl)-acetamide;
5-(2,5-Difluoro-phenyl)-3-oxo-1,3-dihydro-isobenzofuran-1-carboxylic acid [3-(7-fluoro-2-oxo-2H-quinolin-1-yl)-propyl]-amide;
5-Cyclohex-1-enyl-3-oxo-1,3-dihydro-isobenzofuran-1-carboxylic acid [3-(7-fluoro-2-oxo-2H-quinolin-1-yl)-propyl]-amide;
N-[2-(5-Cyclohex-1-enyl-3-oxo-1,3-dihydro-isobenzofuran-1-yl)-ethyl]-2-(7-fluoro-2-oxo-2H-quinolin-1-yl)-acetamide;
N-[2-(5-Cyclopent-1-enyl-3-oxo-1,3-dihydro-isobenzofuran-1-yl)-ethyl]-2-(7-fluoro-2-oxo-2H-quinolin-1-yl)-acetamide;
N-{2-[5-(3,5-Difluoro-phenyl)-3-oxo-1,3-dihydro-isobenzofuran-1-yl]-ethyl}-2-(7-fluoro-2-oxo-2H-quinolin-1-yl)-acetamide; and
2-(7-Fluoro-2-oxo-2H-quinolin-1-yl)-N-{2-[5-(2-methyl-propenyl)-3-oxo-1,3-dihydro-isobenzofuran-1-yl]-ethyl}-acetamide.

12) The invention, thus, relates to compounds of the formula I as defined in embodiment 1), or to such compounds further limited by the characteristics of any one of embodiments 2) to 11), under consideration of their respective dependencies; to pharmaceutically acceptable salts thereof; and to the use of such compounds as medicaments especially for the prevention or treatment of a bacterial infection as set out in the description. Especially the following embodiments relating to the compounds of formula I are thus possible and intended and herewith specifically disclosed in individualized form:

1, 2+1, 3+1, 4+1, 5+1, 6+1, 6+2+1, 6+3+1, 6+4+1, 7+1, 7+2+1, 7+3+1, 7+4+1, 8+1, 8+2+1, 8+3+1, 8+4+1, 8+5+1, 8+6+1, 8+6+2+1, 8+6+3+1, 8+6+4+1, 8+7+1, 8+7+2+1, 8+7+3+1, 8+7+4+1, 9+1, 9+2+1, 9+3+1, 9+4+1, 9+5+1, 9+6+1, 9+6+2+1, 9+6+3+1, 9+6+4+1, 9+7+1, 9+7+2+1, 9+7+3+1, 9+7+4+1, 9+8+1, 9+8+2+1, 9+8+3+1, 9+8+4+1, 9+8+5+1, 9+8+6+1, 9+8+6+2+1, 9+8+6+3+1, 9+8+6+4+1, 9+8+7+1, 9+8+7+2+1, 9+8+7+3+1, 9+8+7+4+1, 10+1, 10+2+1, 10+3+1, 10+4+1, 10+5+1, 10+6+1, 10+6+2+1, 10+6+3+1, 10+6+4+1, 10+7+1, 10+7+2+1, 10+7+3+1, 10+7+4+1, 10+8+1, 10+8+2+1, 10+8+3+1, 10+8+4+1, 10+8+5+1, 10+8+6+1, 10+8+6+2+1, 10+8+6+3+1, 10+8+6+4+1, 10+8+7+1, 10+8+7+2+1, 10+8+7+3+1, 10+8+7+4+1.

In the list above, the numbers refer to the embodiments according to their numbering provided hereinabove whereas “+” indicates the dependency from another embodiment. The different individualized embodiments are separated by commas. In other words, “8+6+3+1” for example refers to embodiment 8) depending on embodiment 6), depending on embodiment 3), depending on embodiment 1), i.e. embodiment “8+6+3+1” corresponds to the compounds of embodiment 1) further limited by the features of the embodiments 3), 6), and 8).

13) A further embodiment of the invention relates to compounds of formula I according to embodiment 1), which are also compounds of formula II

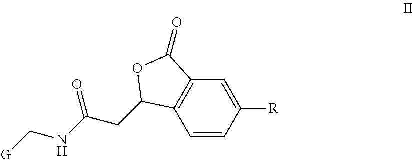

wherein

R represents phenyl optionally substituted with one or two halogen (notably fluorine); cyclopenten-1-yl or cyclohexen-1-yl; or (C₂-C₅)alkenyl; and

G represents:

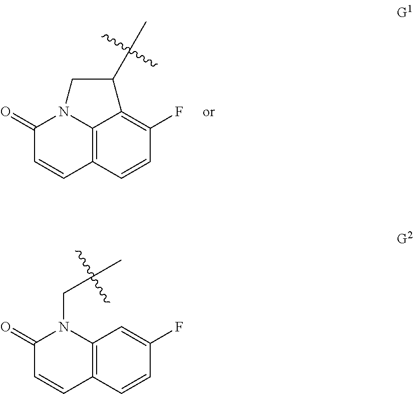 [SMILES: *C1Cn2c(=O)ccc3ccc(F)c1c32 ; *Cn1c(=O)ccc2ccc(F)cc21]

wherein the characteristics of embodiments 2) to 10), especially embodiments 8), 9) and/or 10) apply mutatis mutandis also to the compounds of formula II; i.e the following embodiments relating to the compounds of formula II are thus possible and intended and herewith specifically disclosed in individualized form: 13, 13+8, 13+8+9, 13+8+10, 13+9, 13+10; wherein in the list above, the numbers refer to the embodiments according to their numbering provided hereinabove whereas “+” indicates the dependency from another embodiment as outlined before.

Any reference to a compound of formula I is to be understood as referring also to the salts, especially the pharmaceutically acceptable salts of such a compound, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example “Handbook of Pharmaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quéré (Eds.), RSC Publishing, 2012.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C. The term “room temperature” as used herein refers to a temperature of about 25° C.

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

The present invention also includes isotope labeled, especially ²H (deuterium) labeled compounds of formula I as defined in any one of embodiments 1) to 13) which compounds are identical to the compounds of formula I except that one or more atoms have each been replaced by an atom having the same atomic number but an atomic mass different from the atomic mass usually found in nature. Isotope labeled, especially 2H (deuterium) labeled compounds of formula I and salts thereof are within the scope of the present invention.

Substitution of hydrogen with the heavier isotope 2H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of formula I are not isotope labeled, or they are labeled only with one or more deuterium atoms. In a sub-embodiment, the compounds of formula I are not isotope labeled at all. Isotope labeled compounds of formula I may be prepared in analogy to the methods described hereinafter, but using the appropriate isotope variation of suitable reagents or starting materials.

The compounds formula I as defined in any one of embodiments 1) to 13) and their pharmaceutically acceptable salts can be used as medicaments, e.g. in the form of pharmaceutical compositions for enteral (such especially oral) or parenteral administration (including topical application or inhalation).

In a preferred embodiment of the invention, the administered amount of compound of formula I as defined in any one of embodiments 1) to 13) will be comprised between 1 mg and 2000 mg per day, particularly between 50 mg and 1500 mg per day, more particularly between 100 mg and 1000 mg per day, especially between 250 mg and 1000 mg per day.

A further aspect of the invention are pharmaceutical compositions comprising a compound of formula I as defined in any one of embodiments 1) to 13), or a pharmaceutically acceptable salt thereof, and a pharmaceutically acceptable excipient/carrier material. A pharmaceutical composition according to the present invention contains at least one compound of formula I (or a pharmaceutically acceptable salt thereof) as the active agent, and may also contain additional known antibiotics.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula I or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The compounds of formula I according to the invention, i.e. a compound of formula I as defined in any one of embodiments 1) to 13) above, are suitable for the use as chemotherapeutic active compounds in human and veterinary medicine and as substances for preserving inorganic and organic materials in particular all types of organic materials for example polymers, lubricants, paints, fibres, leather, paper and wood.

The compounds of formula I as defined in any one of embodiments 1) to 13) exhibit antibacterial activity in particular against Gram-positive organisms, but also against Gram-negative pathogens involved in respiratory tract infections (such as especially Haemophilus influenza and Moraxella catarrhalis). They may be used to treat bacterial infections in mammals, especially humans. The compounds may also be used for veterinary applications, such as treating infections in livestock and companion animals, including pigs, ruminants, horses, dogs, cats and poultry.

The compounds of formula I as defined in any one of embodiments 1) to 13) may be used against bacteria and bacteria-like organisms. They may therefore be particularly suitable in human and veterinary medicine for the prophylaxis and chemotherapy of local and systemic infections caused by these pathogens as well as disorders related to bacterial infections. Particular bacterial infections include respiratory tract infections, otitis media, meningitis, skin and soft tissue infections (whether complicated or uncomplicated), pneumonia (including hospital acquired pneumonia), sexually transmitted infections, bacteremia, endocarditis, foreign body infections, osteomyelitis, topical infections, opthalmological infections and tuberculosis. In a sub-embodiment such bacterial infections are respiratory tract infections, or skin and soft tissue infections (whether complicated or uncomplicated).

Bacterial infections may be related to infection by Haemophilus influenzae, Moraxella catarrhalis, Legionella pneumophila, Chlamydia pneumoniae, Chlamydia trachomatis, Actinobacillus haemolyticum, Bartonella henselae, Haemophilus ducreyi, Treponema pallidum, Neiserria gonorrhoeae, Helicobacter pylori, Borrelia recurrentis, Borrelia burgdorferi, Campylobacter jejuni, Bacteroides spp., Bordetella pertussis, Staphylococcus aureus, coagulase-negative staphylococci (i.e., S. epidermidis, S. haemolyticus, etc.), Enterococcus spp., Enterococcus faecalis, E. faecium, E. casseliflavus, E. durans, Peptostreptococcus spp., Streptococcus pneumoniae, Streptococcus pyogenes, Group A to G streptococci, Streptococcus agalactiae, viridans streptococci, Corynebacterium diphtheriae, Corynebacterium minutissimum, Ureaplasma urealyticum, Listeria spp., Mycoplasma pneumonia, Mycobacterium avium, Mycobacterium intracellulare, Mycobacterium tuberculosis, M. leprae, M. paratuberculosis, M. kansasii, M. chelonei, Cryptosporidium spp Clostridium spp., and/or Clostridium perfringens; including strains resistant to known antibacterials such as, but not limited to, beta-lactams, vancomycin, aminoglycosides, quinolones, chloramphenicol, tetracyclines and macrolides.

Bacterial infections (and especially the above-listed particular bacterial infections) may notably be related to infection by S. aureus, Enterococcus faecium, S. pneumonia, streptococci, M. catarrhalis, H. influenzae, and Legionella pneumophila; including strains resistant to known antibacterials such as, but not limited to, beta-lactams, vancomycin, aminoglycosides, quinolones, chloramphenicol, tetracyclines and macrolides.

In particular, the compounds of formula I according to any one of embodiments 1) to 13) or the pharmaceutically acceptable salts thereof, may be used for the preparation of a medicament, and are suitable, for the prevention or treatment of a bacterial infection selected from respiratory tract infections (involving especially S. pneumonia, M catarrhalis and H. influenzae), otitis media (involving especially S. pneumonia, M catarrhalis and H. influenzae), meningitis, skin and soft tissue infections (whether complicated or uncomplicated; involving especially S. aureus and streptococci), pneumonia (including hospital acquired pneumonia; involving especially S. aureus, S. pneumonia, Legionella pneumophila, M. catarrhalis and H. influenzae) and bacteremia.

Furthermore, the compounds of formula I according to any one of embodiments 1) to 13) or the pharmaceutically acceptable salts thereof, may especially be used for the preparation of a medicament, and are suitable, for the prevention or treatment of a bacterial infection mediated by Staphylococcus aureus bacteria or Enterococcus faecium bacteria, especially by quinolone-resistant Staphylococcus aureus bacteria, or Enterococcus faecium quinolone- and vancomycin-resistant bacteria.

The preceding lists of infections and pathogens are to be interpreted merely as examples and in no way as limiting.

Other bacterial infections and disorders related to infections that may be treated or prevented in accord with the method of the present invention are referred to in J. P. Sanford et al., “The Sanford Guide to Antimicrobial Therapy,” 26th Edition, (Antimicrobial Therapy, Inc., 1996).

The compounds of formula I can be manufactured in accordance with the present invention using the procedures described hereafter.

Whenever the compounds of formula I are obtained in the form of mixtures of stereoisomers such as especially enantiomers, the stereoisomers can be separated using methods known to one skilled in the art: e.g. by formation and separation of diastereomeric salts or by HPLC over a chiral stationary phase such as a Daicel ChiralPak AD-H (5 μm) column, a Daicel ChiralCel OD-H (5 μm) column, a Daicel ChiralCel OD (10 μm) column, a Daicel ChiralPak IA (5 μm) column, a Daicel ChiralPak IB (5 μm) column, a Daicel ChiralPak IC (5 μm) column, or a (R,R)-Whelk-01 (5 μm) column. Typical conditions of chiral HPLC are an isocratic mixture of eluent A (EtOH, in presence or absence of a base like triethylamine and/or diethylamine or of an acid like TFA) and eluent B (heptane).

### Preparation of the Compounds of Formula I

### Abbreviations:

The following abbreviations are used throughout the specification and the examples:
Ac acetyl
AcOH acetic acid
AIBN azobisisobutyronitrile
aq. aqueous
Boc tert-butoxycarbonyl
Bs bosyl (benzenesulfonyl)
BuLi n-butyl lithium
CC column chromatography over silica gel
Cipro ciprofloxacin
Cy cyclohexyl
DAD diode array detection
dba dibenzylideneacetone
DCC N,N′-dicyclohexylcarbodiimide
DCE 1,2-dichloroethane
DCM dichloromethane
DIPEA disopropylethylamine
DME dimethylether
DMF N,N-dimethylformamide
DMSO dimethylsulfoxide
DPPA diphenylphosphoryl azide
EA ethyl acetate
EDC N-(3-dimethylaminopropyl)-N′-ethylcarbodiimide
ELSD evaporative light scattering detector
ESI electron spray ionisation
eq. equivalent
Et ethyl
EtOH ethanol
HATU 1-[bis(dimethylamino)methylene]-1H-1,2,3-triazolo[4,5-b]pyridinium 3-oxid hexafluorophosphate
Hept heptane
Hex hexane
HexLi n-hexyl lithium
HOBT 1-hydroxybenzotriazole hydrate
HPLC high pressure liquid chromatography
HV high vacuum conditions
LC liquid chromatography
Me methyl
MeCN acetonitrile
MeOH methanol
MS mass spectroscopy
Ms methanesulfonyl (mesyl)
Ns nosyl (4-nitrophenylsulfonyl)
org. organic
PEPPSI™-IPr [1,3-bis(2,6-diisopropylphenyl)imidazol-2-ylidene](3-chloropyridyl)palladium(II) dichloride
Ph phenyl
Pyr pyridine
Q-Phos 1,2,3,4,5-pentaphenyl-1′-(di-tert-butylphosphino)ferrocene
rt room temperature
sat. saturated
SK-CC01-A 2′-(dimethylamino)-2-biphenylyl-palladium(II) chloride dinorbornylphosphine complex
S-Phos 2-Dicyclohexylphosphino-2′,6′-dimethoxybiphenyl
TBAF tetra-n-butylammonium fluoride
TBDMS tert-butyldimethylsilyl
TBDPS tert-butyldiphenylsilyl
tBu tert-butyl
TEA triethylamine
Tf trifluoromethanesulfonyl (triflyl)
TFA trifluoroacetic acid
THF tetrahydrofuran
TLC thin layer chromatography
t_{R} retention time
Ts para-toluenesulfonyl
T3P 2,4,6-tripropyl-1,3,5,2,4,6-trioxatriphosphorinane-2,4,6-trioxide solution or n-propylphosphonic cyclic anhydride (50% in EA)
XantPhos 4,5-bis(diphenylphosphino)-9,9-dimethylxanthene

### General Reaction Techniques:

### General Reaction Technique 1 (Suzuki Coupling):

The aromatic halide (typically a bromide) is reacted with the required boronic acid derivative or its boronate ester equivalent (e.g. pinacol ester) in the presence of a palladium catalyst and a base such as K₂CO₃, Cs₂CO₃, K₃PO₄, tBuONa or tBuOK between 20 and 120° C. in a solvent such as toluene, THF, dioxane, DME or DMF, usually in the presence of water (20 to 50%). Examples of typical palladium catalysts are triarylphosphine palladium complexes such as Pd(PPh₃)₄. These catalysts can also be prepared in situ from a common palladium source such as Pd(OAc)₂ or Pd₂(dba)₃ and a ligand such as trialkylphosphines (e.g. PCy₃ or P(tBu)₃), dialkylphosphinobiphenyls (e.g. S-Phos) or ferrocenylphosphines (e.g. Q-Phos). Alternatively, one can use a commercially available precatalyst based on palladacycle (e.g. SK-CC01-A) or N-heterocyclic carbene complexes (e.g. PEPPSI™-IPr). The reaction can also be performed by using the corresponding aromatic triflate. Further variations of the reaction are described in Chem. Rev. (1995), 95, 2457-2483, Synthesis (2004), 2419-2440, Aldrichimica acta (2006), 39, 17-24 and 97-111, Acc. Chem. Res. (2008), 41, 1555-1564, and references cited therein.

### General Reaction Technique 2 (Amide Coupling):

The carboxylic acid is reacted with the amine in presence of an activating agent such as DCC, EDC, HOBT, n-propylphosphonic cyclic anhydride (T3P), HATU, or di-(N-succinimidyl)-carbonate, in presence of a base such as TEA or DIEPA, in a dry aprotic solvent such as EA, DCM, MeCN or DMF between −20° C. and 60° C. (see G. Benz in Comprehensive Organic Synthesis, B. M. Trost, I. Fleming, Eds; Pergamon Press: New York (1991), vol. 6, p. 381). Alternatively, the carboxylic acid can be activated by conversion into its corresponding acid chloride by reaction with oxalyl chloride or thionyl chloride neat or in a solvent like DCM between −20° and 60° C. Further activating agents can be found in Comprehensive Organic Transformations. A guide to Functional Group Preparations; 2^{nd} Edition, R. C. Larock, Wiley-VC; New York, Chichester, Weinheim, Brisbane, Singapore, Toronto, 1999. Section nitriles, carboxylic acids and derivatives p. 1941-1949.

### General Reaction Technique 3 (Reductive Amination):

The reaction between the amine and the aldehyde or ketone is performed in a solvent system allowing the removal of the formed water through physical or chemical means (e.g. distillation of the solvent-water azeotrope or presence of drying agents such as molecular sieves, MgSO₄ or Na₂SO₄). Such solvent is typically toluene, Hex, THF, DCM or DCE or a mixture of solvents such as DCE/MeOH. The reaction can be catalyzed by traces of acid (usually AcOH). The intermediate imine is reduced with a suitable reducing agent (e.g. NaBH₄, NaBH₃CN, or NaBH(OAc)₃ or through hydrogenation over a noble metal catalyst such as Pd/C. The reaction is carried out between −10° C. and 110° C., preferably between 0° C. and 60° C. The reaction can also be carried out in one pot. It can also be performed in protic solvents such as MeOH or water in presence of a picoline-borane complex (Sato et al., Tetrahedron (2004), 60, 7899-7906).

### General Reaction Technique 4 (Amine Substitution):

The amine derivative is reacted with the corresponding sulfonate derivative (or its corresponding iodide) in presence of an inorganic base such as K₂CO₃ or an organic base such as TEA in a solvent such as THF between 0° C. and 80° C. Further details can be found in Comprehensive Organic Transformations. A guide to Functional Group Preparations; 2^{nd} Edition, R. C. Larock, Wiley-VC; New York, Chichester, Weinheim, Brisbane, Singapore, Toronto, 1999. Section Amines p. 779.

### General Reaction Technique 5 (Alcohol Activation):

The alcohol is reacted with MSCl, TfCl, BsCl, NsCl or TsCl in presence of a base such as TEA in a dry aprotic solvent such as Pyr, THF or DCM between −30° C. and +50° C. In the case of the triflate or mesylate, Tf₂O or Ms₂O can also be used.

### General Reaction Technique 6 (Formation of Iodo, Chloro or Bromo Derivatives and Subsequent Corresponding Azide):

The sulfonates obtained using general reaction technique 5 can be reacted with a sodium halogenide such as NaI or NaBr in MeCN or DMF between 40° C. and 120° C. delivering the corresponding iodide derivatives. Alternatively the corresponding bromides or chlorides can also be obtained by reaction of the corresponding alcohol derivatives with PBr₃ or PCl₃ respectively.

General Reaction Technique 7 (Azide into Amine):

The azides are hydrogenated over a noble metal catalyst such as Pd/C in solvent such as MeOH or EA. Alternatively the reduction can be performed using PPh₃ in presence of water as described in J. Med. Chem. (1993), 36, 2558-68.

### General Reaction Technique 8 (Removal of Hydroxy Protecting Groups):

The silyl ether groups are removed either using fluoride anion sources such as TBAF in THF between 0° C. and +40° C. or HF in MeCN or water between 0° C. and +40° C. or using acidic conditions such as AcOH in THF/MeOH or HCl in MeOH. Further methods to remove the TBDMS and TBDPS groups are given in T. W. Greene, P. G. M. Wuts, Protecting Groups in Organic Synthesis, 3rd Ed. (1999), 133-139 and 142-143 respectively (Publisher: John Wiley and Sons, Inc., New York, N.Y.). Further general methods to remove alcohol protecting groups are described in T. W. Greene, P. G. M. Wuts, Protecting Groups in Organic Synthesis, 3rd Ed. (1999), 23-147 (Publisher: John Wiley and Sons, Inc., New York, N.Y.).

### General Reaction Technique 9 (Alcohol Oxidation):

The alcohol dissolved in an organic solvent such as DCM or THF is oxidized into the corresponding aldehyde with MnO₂. Further methods can be found in Comprehensive Organic Transformations. A guide to Functional Group Preparations; 2^{nd} Edition, R. C. Larock, Wiley-VC; New York, Chichester, Weinheim, Brisbane, Singapore, Toronto, 1999; Section aldehydes and ketones, p. 1234-1236.

### General Preparation Methods:

### Preparation of the Compounds of Formula I:

The compounds of formula I can be manufactured by the methods given below, by the methods given in the examples or by analogous methods. Optimum reaction conditions may vary with the particular reactants or solvents used, but such conditions can be determined by a person skilled in the art by routine optimisation procedures.

Sections hereafter describe general methods for preparing compounds of formula I. If not indicated otherwise, the generic groups A, M, M¹, M¹⁻¹, M¹⁻², M² R, R¹, R², U and V are as defined for formula I. General synthetic methods used repeatedly throughout the text below are referenced to and described in the above section entitled “General reaction techniques”. In some instances certain generic groups might be incompatible with the assembly illustrated in the procedures and schemes below and so will require the use of protecting groups. The use of protecting groups is well known in the art (see for example “Protective Groups in Organic Synthesis”, T. W. Greene, P. G. M. Wuts, Wiley-Interscience, 1999).

The compounds of formula I can be obtained by:

a) reacting a compound of structure II

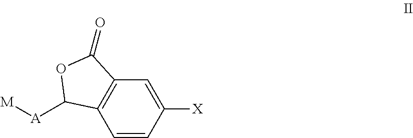

wherein X represents a halogen such as iodine or bromine with a compound of structure III (or an ester thereof)

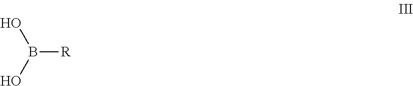

using general reaction technique 1.

b) reacting a compound of structure IV

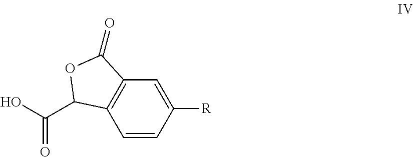

with a compound of structure V

M-Q-NH₂ V

wherein either M represents M¹⁻¹ or M¹⁻² and Q represents —NH—CH₂—CH₂— or M represents M² and Q represents —CH₂—CH₂— or —CH₂—CH₂—CH₂—, using general reaction technique 2.

c) reacting a compound of structure VI

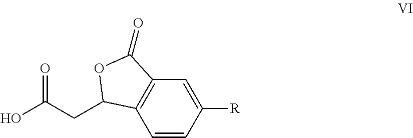

with a compound of structure VII

M-Q-NH₂ VII

wherein either M represents M¹⁻¹ or M¹⁻² and Q represents —CH₂— or M represents M² and Q represents —CH₂—CH₂—, using general reaction technique 2.

d) reacting a compound of structure VIII

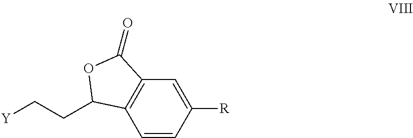

wherein Y represents either OSO₂R^{a} wherein R^{a} represents (C₁-C₁₀)alkyl, trifluoromethyl of tolyl, or a halogen such as iodine or bromine, with a compound of structure IX

M-Q-NH₂ IX

wherein either M represents M¹⁻¹ or M¹⁻² and Q represents —CH₂— or M represents M² and Q represents —CH₂—CH₂—, using general reaction technique 4.

e) reacting a compound of structure X

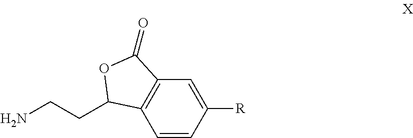

with a compound of structure XI

M-Q-COOH XI

wherein M represents M² and Q represents —CH₂—, using general reaction technique 2.

f) reacting a compound of structure XII

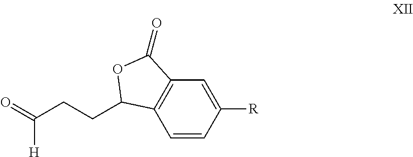

with a compound of structure XIII

M-NH₂ XIII

wherein M represents M¹⁻¹ or M¹⁻² using general reaction technique 3.

### Preparation of the Synthesis Intermediates:

### Compounds of Structure II:

Compounds of structure II can be obtained by
aa) reacting a compound of structure XIV

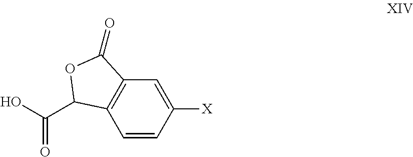

wherein X represents halogen such as bromine or iodine, with a compound of structure V

M-Q-NH₂ V

wherein either M represents M¹⁻¹ or M¹⁻² and Q represents —NH—CH₂—CH₂— or M represents M² and Q represents —CH₂—CH₂—CH₂—, using general reaction technique 2.
bb) reacting a compound of structure XV

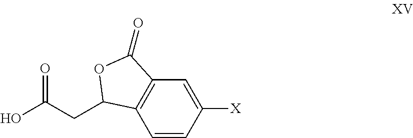

wherein X represents halogen such as bromine or iodine, with a compound of structure VII

M-Q-NH₂ VII

wherein either M represents M¹⁻¹ or M¹⁻² and Q represents —CH₂— or M represents M² and Q represents —CH₂—CH₂—, using general reaction technique 2.
cc) reacting a compound of structure XVI

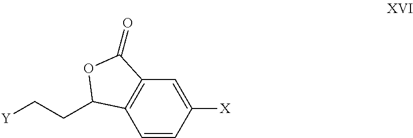

wherein X represents halogen such as bromine or iodine, Y represents either OSO₂R^{a} wherein R^{a} represents (C₁-C₁₀)alkyl, trifluoromethyl, phenyl, 4-nitrophenyl or tolyl, or a halogen such as iodine or bromine, with a compound of structure IX

M-Q-NH₂ IX

wherein M represents M² and Q represents —CH₂—CH₂—, using general reaction technique 4.
dd) reacting a compound of structure XVII

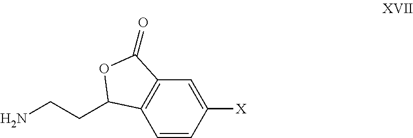

wherein X represents halogen such as bromine or iodine, with a compound of structure XI

M-Q-COOH XI

wherein M represents M² and Q represents —CH₂—, using general reaction technique 2.
ee) reacting a compound of structure XVIII

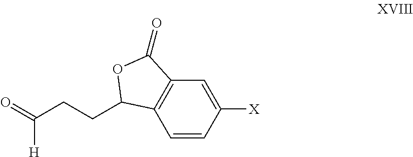

wherein X represents halogen such as bromine or iodine, with a compound of structure XIII

M-NH₂ XIII

wherein M represents M¹⁻¹ or M¹⁻² using general reaction technique 3.

### Compounds of Formulae IV and XIV:

The compounds of structures IV and XIV can be prepared as summarised in Scheme 1 hereafter.

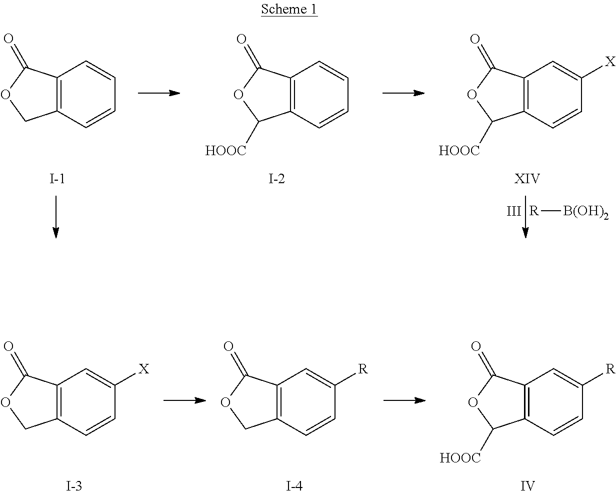

In Scheme 1, X represents a halogen such as bromine or iodine.

Phthalide (commercial) is deprotonated using LDA and quenched with CO₂ to obtain the intermediate I-2 which is brominated with NBS to give derivatives XIV (X═Br).

Compound of structure XIV is reacted with the commercially available boronic acid derivatives of structure III, using general reaction technique 1, affording the compounds of structure IV.

Alternatively, the compound of structure I-1 can be brominated with NBS and the resulting derivative of structure I-3 (X═Br) is reacted with the commercially available boronic acid derivatives of structure III, using general reaction technique 1, affording the intermediates of structure I-4 which can be transformed into the derivatives of formula IV by deprotonation using LDA and quenching with CO₂.

### Compounds of Structures VI, VIII, X and XV-XVII:

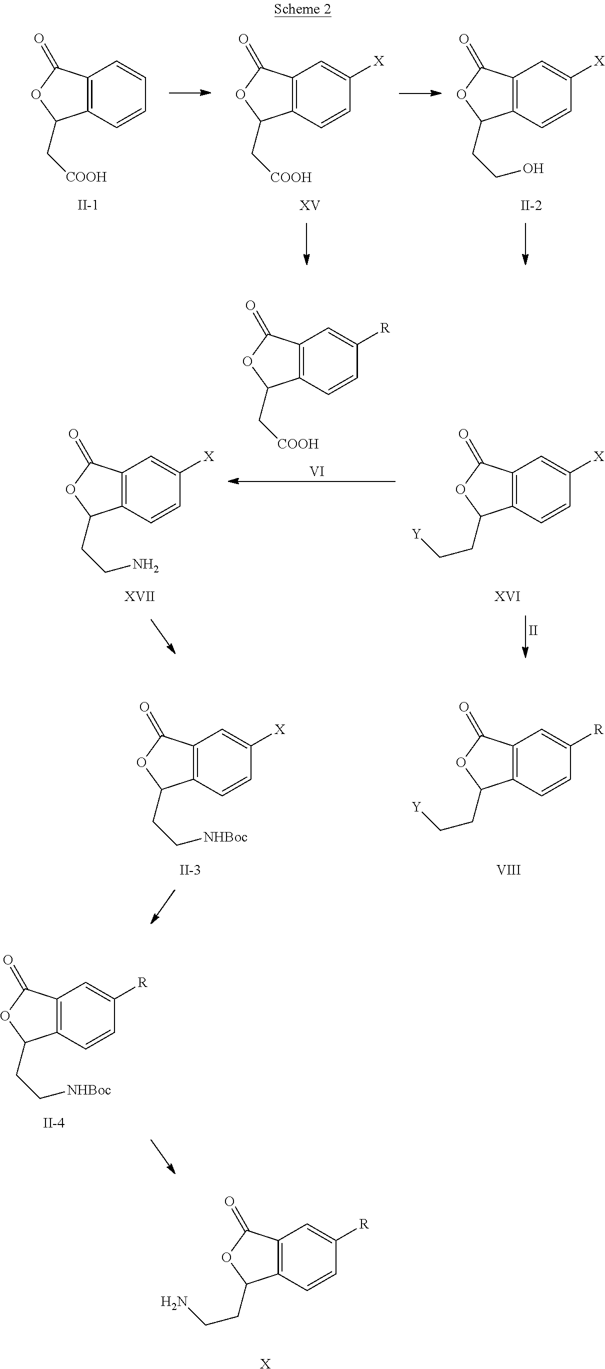

In Scheme 2, X represents a halogen such as bromine, iodine and Y represents a halogen such as bromine or iodine or the group OSO₂R^{a} wherein R^{a} represents (C₁-C₁₀)alkyl, trifluoromethyl, phenyl, 4-nitrophenyl or tolyl.

3-Phthalideacetic acid (commercial; CAS 4743-58-2) can be reacted with NBS affording the intermediate of structure XV wherein X represents Br. The latter may be reacted with the boronic acid derivatives of structure III, using general reaction technique 1, affording the intermediates of structure VI. The intermediate of structure XV can be reduced with BH₃ into the alcohol of structure II-2. The latter can be sequentially transformed into the derivatives of structure XVI wherein Y is OSO₂R^{a}, using general reaction technique 5 and Y is iodine, using general reaction technique 6. The intermediates of structure XVI wherein Y represents iodine or OSO₂R^{a} can be reacted with the boronic acid derivatives of structure III, using general reaction technique 1, affording the intermediates of structure VIII. Alternatively, compounds of structure VIII can be obtained from the compound of structure II-2 (X═Br) by reaction with the boronic acid derivatives of structure III, using general reaction technique 1, followed by sequential transformation into the derivatives of structure VIII wherein Y is OSO₂R^{a}, using general reaction technique 5 and Y is iodine, using general reaction technique 6. The intermediates of structure XVI may be transformed into the corresponding azido derivatives by reaction with sodium azide, followed by reduction of the azido intermediates into the corresponding amine derivatives of structure XVII, using general reaction technique 7. The latter can be reacted with Boc₂O, affording the intermediate derivatives of formula II-3 which can be reacted with the boronic acid derivatives of structure III, using general reaction technique 1, followed by deprotection of the Boc group using TFA or HCl affording the intermediates of structure X.

### Compounds of Structures XII and XVIII:

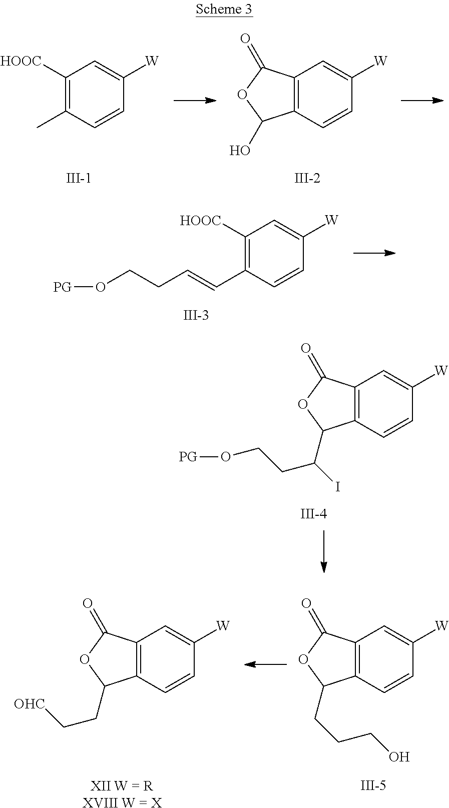

In Scheme 3, W represents R or X, X represents a halogen such as bromine, iodine and PG represents an alcohol protecting group such as TBDMS or TBDPS.

The benzoic acid derivatives of structure III-1 (commercially available when W=halogen, e.g. CAS 54811-38-0 (W═I) or CAS 79669-49-1 (W═Br), or when W═R, prepared by reaction of the compound of structure III-1 wherein W is Br with the compounds of structure III using general reaction technique 1) can be reacted with NBS under light irradiation followed by hydrolysis of the intermediate dibromide, affording the derivatives of structure III-2. The resulting hydroxy-lactone derivatives of structure III-2 may be reacted with [3-[[(1,1-dimethylethyl)dimethylsilyl]oxy]propylidene]triphenyl-phosphorane (CAS 131318-58-6; prepared according to WO 94/20519) affording the derivatives of structure III-3. The latter can be ring closed by iodo-lactonization affording the derivatives of structure III-4 which can be de-iodinated by treatment with tributyl-tin hydride followed by removal of the alcohol protecting group using general reaction technique 8. The resulting derivatives of structure III-5 can be transformed into the corresponding aldehyde derivatives of structures XII and XVIII using general reaction technique 9.

The compounds of structures V, VII, IX wherein M is M² and Q is —CH₂—CH₂—CH₂— or —CH₂—CH₂— can be prepared as described in scheme 4 hereafter.

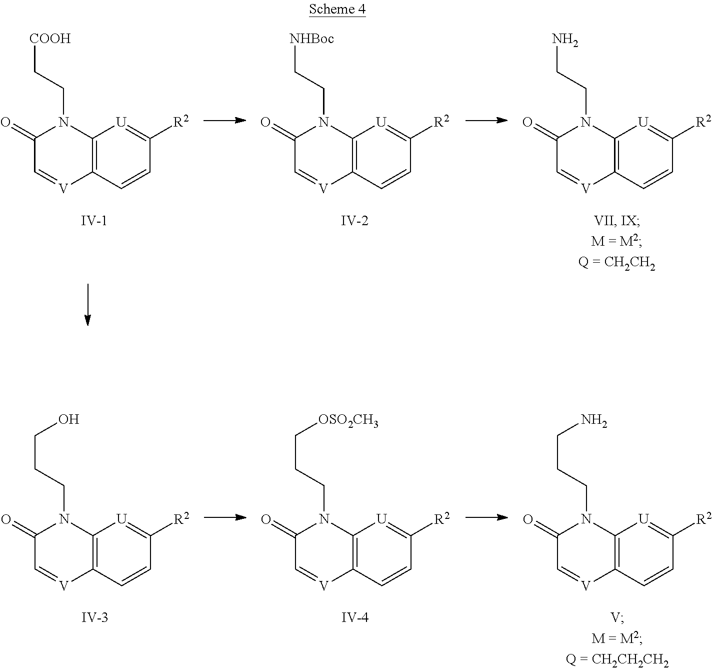

The carboxylic acid derivatives of structure IV-1 can be reacted with DPPA in presence of tBuOH affording the carbamate derivatives of structure IV-2. The Boc protecting group can be removed by treatment with an organic or inorganic acid such as TFA in DCM or HCl in dioxane, affording the derivatives of structures VII or IX wherein M is M² and Q is —CH₂—CH₂—. The carboxylic acid derivatives of structure IV-1 can also be reduced into the corresponding alcohol derivatives of structure IV-3 by treatment with borane in THF. The latter derivatives can be transformed into the corresponding mesylate derivatives of structure IV-4 using general reaction technique 5 and transformed into the corresponding amino derivatives of structure V wherein M is M² and Q is —CH₂—CH₂—CH₂— by reaction with sodium azide followed by reduction using general reaction techniques 6 and 7. The compound of structure V wherein U═V═N can be prepared according to WO2009/104159.

The compounds of structures V, VII, XIII wherein M is M¹⁻¹ or M¹⁻² and Q is a bond or —CH₂— can be prepared according to or in analogy to WO2010/041194 (CAS 1220978-22-2: Q=—CH₂—; CAS 1220980-46-0: Q=bond). The compounds of structure V wherein M is M¹⁻¹ or M¹⁻² and Q is —NH—CH₂—CH₂— can be prepared as described in scheme 5 hereafter.

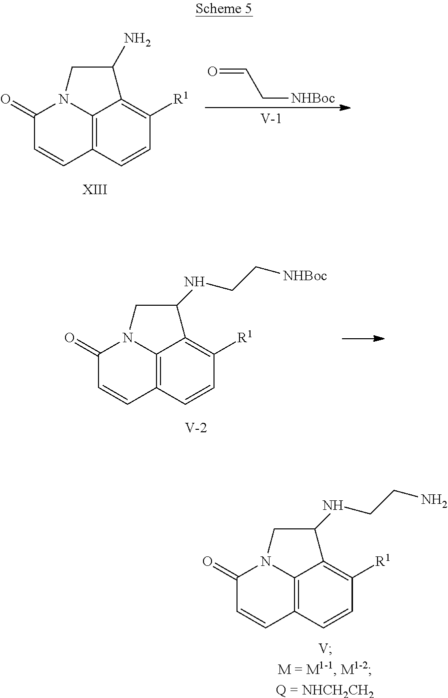

The derivatives of structure XIII, prepared according to WO2010/041194 can be reacted with the commercially available N-(2-oxoethyl)-carbamic acid tBu ester (V-1; CAS 89711-08-0) using general reaction technique 3, affording the derivatives of structure V-2. The latter can be transformed into the corresponding derivatives of structure V wherein M represents M¹⁻¹ or M¹⁻² and Q represents —NH—CH₂—CH₂— by treatment with an organic or inorganic acid such as TFA in DCM or HCl in dioxane.

The compounds of formula XI are commercially available (e.g. CAS 1280736-31-3) or can be prepared according to WO 2011/148962 (7-methoxy-2-oxo-1(2H)-quinoxalineacetic acid; CAS 1351402-50-0 and 7-methoxy-2-oxo-1(2H)-quinolineacetic acid; CAS 951159-87-8), WO 2003/068743 (2-oxo-1(2H)-quinoxaline acetic acid, CAS 63642-41-1).

The compounds of structure IV-1 are commercially available (e.g. 2-oxo-1(2H)-quinoxalinepropanoic acid; CAS 1016760-97-6) or can be prepared according to WO 2008/116815 (7-fluoro-2-oxo-1(2H)-quinolinepropanoic acid, CAS 1065677-04-4) or WO 2009/104159 (7-methoxy-2-oxo-1(2H)-quinolinepropanoic acid, CAS 1185181-94-5).

Particular embodiments of the invention are described in the following Examples, which serve to illustrate the invention in more detail without limiting its scope in any way.

### EXAMPLES

All temperatures are stated in ° C. All temperatures are stated in ° C. Unless otherwise indicated, the reactions take place at rt.

Analytical TLC characterisations were performed with 0.2 mm plates: Merck, Silica gel 60 F₂₅₄. Elution is performed with EA, Hept, DCM, MeOH or mixtures thereof. Detection was done with UV or with a solution of KMnO₄ (3 g), K₂CO₃ (20 g), 5% NaOH (3 mL) and H₂O (300 mL) with subsequent heating.

CCs were performed using Brunschwig 60A silica gel (0.032-0.63 mm), elution being carried out with EA, Hept, DCM, MeOH or mixtures thereof. When the compounds contained an acid function, 1% of AcOH was added to the eluent(s). NH₄OH as used for CC is 25% aq.

Compounds were characterized by ¹H-NMR (300 MHz) (Varian Oxford); or by ¹H-NMR (400 MHz) (Bruker Advance 400). Chemical shifts δ are given in ppm relative to the solvent used; multiplicities: s=singlet, d=doublet, t=triplet, q=quadruplet, p=pentuplet, hex=hexet, hep=heptet, m=multiplet, br.=broad; coupling constants J are given in Hz. Alternatively compounds were characterized by LC-MS (Sciex API 2000 with Agilent 1100 Binary Pump with DAD and ELSD or an Agilent quadrupole MS 6140 with Agilent 1200 Binary Pump, DAD and ELSD); by TLC (TLC plates from Merck, Silica gel 60 F₂₅₄); or by melting point.

The analytical LC-MS data have been obtained using the following respective conditions:
MS1 data:
   Column: Zorbax SB-Aq, 3.5 μm, 4.6×50 mm;
   Injection volume: 1 μL;
   Column oven temperature: 40° C.;
   Detection: UV 210 nm, ELSD and MS;
   MS ionization mode: ESI+;
   Eluents: A: H₂O+0.04% TFA; and B: MeCN;
   Flow rate: 4.5 mL/min;
   Gradient: 5% B to 95% B (0.0 min-1.0 min), 95% B (1.0 min-1.45 min).

The number of decimals given for the corresponding [M+H⁺] peak(s) of each tested compound depends upon the accuracy of the LC-MS device actually used.

### Preparations

### Preparation A: rac-(5-bromo-3-oxo-1,3-dihydro-isobenzofuran-1-yl)-acetic acid

A solution of phthalide-3-acetic acid (2.0 g; commercial; CAS343953-55-9) in TFA (5.2 mL) and H₂SO₄ (2.3 mL) was treated with NBS (2.78 g) and further stirred at rt for 5 d. The reaction mixture was poured into ice-water and extracted with ether. The org. layer was washed with water and brine (3×), dried over MgSO₄ and concentrated under reduced pressure. The residue was redissolved in MeOH and evaporated under reduced pressure, affording after stirring in ether 1.6 g (57% yield) of a colourless solid.

MS1 (ESI, m/z): 311.8 [M+MeCN+H⁺]; t_{R}=0.66 min.

### Preparation B: rac-(3-oxo-5-phenyl-1,3-dihydro-isobenzofuran-1-yl)-acetic acid

A solution of compound of preparation A (1.50 g) in DME (13 mL) and water (4.5 mL) was treated with phenylboronic acid (1.01 g; commercial; CAS 98-80-6) and Pd(PPh₃)₄ (320 mg) and K₂CO₃ (1.53 g). The reaction mixture was stirred for 1.5 h at 90° C., allowed to cool to rt, diluted with water and extracted with EA. The pH of the aq. layer was adjusted to pH1 by addition of 1N HCl and extracted with DCM. The combined org layers were dried over MgSO₄ and concentrated under reduced pressure. The residue was purified by CC (Hept/EA 1:1 to DCM/MeOH 4:1), affording 1.1 g (74% yield) of a yellow foam.

MS1 (ESI, m/z): 310.0 [M+MeCN+H⁺]; t_{R}=0.76 min.

### Preparation C: 2-(3-oxo-5-phenyl-1,3-dihydroisobenzofuran-1-yl)ethyl methanesulfonate

### C.i. rac-3-(2-hydroxy-ethyl)-6-phenyl-3H-isobenzofuran-1-one

A solution of compound of preparation B (900 mg) in THF (30 mL) was cooled to 2° C. and treated dropwise with a solution of BH₃ in THF (1M, 10 mL) and further stirred at rt for 2 h. The reaction mixture was quenched with MeOH and the solvents were removed under reduced pressure. The residue was purified by CC (Hept/EA 2:1 to 0:1), affording 620 mg (73% yield) of a colourless oil.

MS1 (ESI, m/z): 296.1 [M+MeCN+H⁺]; t_{R}=0.76 min.

### C.ii. -(3-oxo-5-phenyl-1,3-dihydroisobenzofuran-1-yl)ethyl methanesulfonate

A solution of intermediate C.i. (310 mg) in DCM (10 mL) was treated with TEA (0.42 mL) and MsCl (0.10 mL) and further stirred at rt for 30 min. The reaction mixture was diluted with DCM and water. The org phase was dried over MgSO₄ and concentrated under reduced pressure, affording 0.46 g (100% yield) of a yellow oil.

MS1 (ESI, m/z): 333.1 [M+H⁺]; t_{R}=0.85 min.

### Preparation D: rac-methanesulfonic acid 2-(5-bromo-3-oxo-1,3-dihydro-isobenzofuran-1-yl)-ethyl ester

### D.i. 6-bromo-3-(2-hydroxyethyl)isobenzofuran-1(3H)-one

A solution of compound of preparation A (5.0 g) in THF (165 mL) was cooled to 2° C. and treated dropwise with a solution of BH₃ in THF (1M, 40 mL) and further stirred at rt for 3 h. The reaction mixture was quenched with MeOH and the solvents were removed under reduced pressure. The residue was purified by CC (Hept/EA 1:1 to 0:1), affording 3.80 g (80% yield) of a colourless oil.

MS1 (ESI, m/z): 300.0 [M+MeCN+H⁺]; t_{R}=0.66 min.

### D.ii. rac-methanesulfonic acid 2-(5-bromo-3-oxo-1,3-dihydro-isobenzofuran-1-yl)-ethyl ester

Starting from intermediate D.i (3.80 g) and MsCl (1.26 mL) and proceeding in analogy to preparation C, step C.ii, the title compound was obtained as a beige solid (4.8 g; 97% yield). MS1 (ESI, m/z): 377.9 [M+MeCN+H⁺]; t_{R}=0.77 min.

### Preparation E: rac-methanesulfonic acid 2-[5-(2,4-difluoro-phenyl)-3-oxo-1,3-dihydro-isobenzofuran-1-yl]-ethyl ester

Starting from compound of preparation D (500 mg) and 2,4-difluorophenylboronic acid (353 mg; commercial; CAS 144025-03-6) and proceeding in analogy to preparation B, the title compound was obtained as a beige solid (176 mg; 32% yield).

MS1 (ESI, m/z): 369.1 [M+H⁺]; t_{R}=0.87 min.

### Preparation F: rac-[2-(5-bromo-3-oxo-1,3-dihydro-isobenzofuran-1-yl)-ethyl]-carbamic acid tert-butyl ester

### F.i. 3-(2-azidoethyl)-6-bromoisobenzofuran-1(3H)-one

A solution of compound of preparation D (5.50 g) in DMF (25 ml) was heated at 80° C. for 1 h in presence of NaN₃ (1.28 g). The reaction mixture was diluted with water and extracted with EA. The org. layer was washed with water/brine (3×), dried over MgSO₄, concentrated under reduced pressure and purified by CC (Hept/EA 2:1), affording 3.4 g (74% yield) of a light yellow oil.

MS1 (ESI, m/z): 325.1 [M+MeCN+H⁺]; t_{R}=0.85 min.

### F.ii. rac-[2-(5-bromo-3-oxo-1,3-dihydro-isobenzofuran-1-yl)-ethyl]-carbamic acid tert-butyl ester

A solution of intermediate F.i. (3.40 g) in THF (70 mL) and water (2.7 mL) was treated with PPh₃ (3.48 g) and further stirred at 50° C. for 2 h. The reaction mixture was cooled to rt, treated with BOC₂O (3.94 g) and further stirred at rt for 2 h. The reaction mixture was evaporated under reduced pressure and the residue was stirred in EA/Hept and filtered. The filtrate was evaporated under reduced pressure and purified by CC (Hept/EA 1:1 to 0:1), affording 2.78 g (65% yield) of a colourless oil.

MS1 (ESI, m/z): 713.3 [2M+H⁺]; t_{R}=0.87 min.

### Preparation G: 3-(2-aminoethyl)-6-(cyclohex-1-en-1-yl)isobenzofuran-1(3H)-one hydrochloride

### G.i. tert-butyl (2-(5-(cyclohex-1-en-1-yl)-3-oxo-1,3-dihydroisobenzofuran-1-yl)ethyl)carbamate

Starting from compound of preparation F (300 mg) and 1-cyclohexenylboronic acid (159 mg; commercial; CAS 89490-05-1) and proceeding in analogy to preparation B, the title compound was obtained as a colourless oil (238 mg; 79% yield).

MS1 (ESI, m/z): 299.3 [M-Boc+MeCN+H⁺]; t_{R}=0.98 min.

### G.ii. 3-(2-aminoethyl)-6-(cyclohex-1-en-1-yl)isobenzofuran-1(3H)-one hydrochloride

A solution of intermediate G.i (220 mg) in dioxane (1 mL) was treated with 4M HCl in dioxane (1.54 ml) and further stirred at rt for 3 h. The reaction mixture was diluted with ether and the solid was collected by filtration affording 140 mg (77% yield; hydrochloride) of a colourless solid.

MS1 (ESI, m/z): 299.3 [M+MeCN+H⁺]; t_{R}=0.65 min.

### Preparation H: 3-(2-aminoethyl)-6-(cyclopent-1-en-1-yl)isobenzofuran-1(3H)-one hydrochloride

### H.i. tert-butyl (2-(5-(cyclopent-1-en-1-yl)-3-oxo-1,3-dihydroisobenzofuran-1-yl)ethyl)carbamate

Starting from compound of preparation F (300 mg) and 1-cyclopentenylboronic acid (141 mg; commercial; CAS 850036-28-1) and proceeding in analogy to preparation B, the title compound was obtained as a colourless solid (211 mg; 73% yield).

MS1 (ESI, m/z): 285.4 [M-Boc+MeCN+H⁺]; t_{R}=0.96 min.

### H.ii. 3-(2-aminoethyl)-6-(cyclopent-1-en-1-yl)isobenzofuran-1(3H)-one hydrochloride

Starting from intermediate H.i. (200 mg) and proceeding in analogy to preparation G, step G.ii, the title compound was obtained as a colourless solid (146 mg; 90% yield).

MS1 (ESI, m/z): 244.4 [M+H⁺]; t_{R}=0.62 min.

### Preparation I: 3-(2-aminoethyl)-6-(3,5-difluorophenyl)isobenzofuran-1(3H)-one hydrochloride

### I.i. tert-butyl (2-(5-(3,5-difluorophenyl)-3-oxo-1,3-dihydroisobenzofuran-1-yl)ethyl)carbamate

Starting from compound of preparation F (300 mg) and 3,5-difluorophenylboronic acid (199 mg; commercial; CAS 156545-07-2) and proceeding in analogy to preparation B, the title compound was obtained as a colourless oil (235 mg; 72% yield).

MS1 (ESI, m/z): 331.3 [M-Boc+MeCN+H⁺]; t_{R}=0.96 min.

### I.ii. 3-(2-aminoethyl)-6-(3,5-difluorophenyl)isobenzofuran-1(3H)-one hydrochloride

Starting from intermediate I.i. (220 mg) and proceeding in analogy to preparation G, step G.ii, the title compound was obtained as a colourless solid (159 mg; 86% yield).

MS1 (ESI, m/z): 290.3[M+H⁺]; t_{R}=0.64 min.

### Preparation J: rac-3-(2-amino-ethyl)-6-(2-methyl-propenyl)-3H-isobenzofuran-1-one

### J.i. tert-butyl (2-(5-(2-methylprop-1-en-1-yl)-3-oxo-1,3-dihydroisobenzofuran-1-yl)ethyl)carbamate

Starting from compound of preparation F (300 mg) and 2,2-dimethylethenylboronic acid (126 mg; commercial; CAS 14559-88-7) and proceeding in analogy to preparation B, the title compound was obtained as a yellow oil (220 mg; 79% yield).

MS1 (ESI, m/z): 663.6 [2M+H⁺]; t_{R}=0.93 min.

### J.ii. 3-(2-aminoethyl)-6-(3,5-difluorophenyl)isobenzofuran-1(3H)-one

Starting from intermediate J.i. (204 mg) and proceeding in analogy to preparation G, step G.ii, the title compound was obtained as a colourless solid (153 mg; 93% yield).

MS1 (ESI, m/z): 273.3 [M+MeCN+H⁺]; t_{R}=0.60 min.

### Preparation K: 3-(3-oxo-5-phenyl-1,3-dihydroisobenzofuran-1-yl)propanal

### K.i. rac-3-hydroxy-6-phenyl-3H-isobenzofuran-1-one

A suspension of 4-methyl-3-biphenylcarboxylic acid (2.22 g; commercial; CAS 2840-35-9) in CCl₄ (60 mL) was refluxed at 80° C. under irradiation with a 200 W spotlight in presence of NBS (4.1 g). The reaction mixture was cooled to rt and filtered. The filtrate was evaporated under reduced pressure and the residue was suspended in water and refluxed for 2 h. The reaction mixture was extracted with EA (2×). The combined org. layers were washed with brine, dried over MgSO₄, concentrated under reduced pressure, affording after stirring of the residue in MeOH, 1.1 g (46% yield) of a light beige solid.

MS1 (ESI, m/z): 227.2 [M+H⁺]; t_{R}=0.75 min.

### K.ii. 4-[(E)-4-(tert-butyl-dimethyl-silanyloxy)-but-1-enyl]-biphenyl-3-carboxylic acid

A suspension of intermediate K.i (301 mg) and [3-[[(1,1-dimethylethyl)dimethylsilyl]oxy]propyl]triphenyl-phosphonium bromide (1.10 g; commercial; CAS 120379-86-7) in THF (10 mL) was treated portionwise with KOtBu (373 mg). The reaction was stirred at rt for 1 h. The pH of the reaction mixture was adjusted to pH 4 by the addition of 10% aq. citric acid solution and the reaction mixture was extracted with EA. The org. layer was washed with brine, dried over MgSO₄, concentrated under reduced

pressure and purified by CC (EA), affording 452 mg (86% yield) of a yellow oil.

MS1 (ESI, m/z): 384.3 [M+H⁺]; t_{R}=1.06 min.

### rac-3-[3-(tert-butyl-dimethyl-silanyloxy)-1-iodo-propyl]-6-phenyl-3H-isobenzofuran-1-one

A solution of intermediate K.ii (434 mg) in DCM (1.7 mL; was sequentially treated with NaHCO₃ (105 mg), water (5 mL), KI (697 mg) and iodine (346 mg) and further stirred at rt for 3 h. The reaction mixture was treated with excess aq saturated sodium thiosulfate solution and extracted with DCM. The aq. layer was extracted with DCM (3×). The combined org. layers were dried over MgSO₄, concentrated under reduced pressure affording 450 mg (76% yield) of a beige solid.

MS1 (ESI, m/z): 509.2 [M+H⁺]; t_{R}=1.13 min.

### K.iv. 3-(3-((tert-butyldimethylsilyl)oxy)propyl)-6-phenylisobenzofuran-1(3H)-one

A solution of intermediate K.iii (440 mg) in toluene (dry; 7.5 mL) was treated with Bu₃SnH (624 mg) and AIBN (8 mg) and further refluxed for 6 h. The reaction mixture was treated with water and extracted with EA (2×). The combined org. layers were dried over MgSO₄, concentrated under reduced pressure and purified by CC (Hept/EA 4:1) affording 0.44 g (100% yield) of a light yellow oil.

MS1 (ESI, m/z): 383.2 [M+H⁺]; t_{R}=1.10 min.

### K.v. 3-(3-hydroxypropyl)-6-phenylisobenzofuran-1(3H)-one

A solution of intermediate K.iv (329 mg) in THF (10 mL) was treated with 1M TBAF in THF (1.90 mL) and further stirred at rt for 2 h. The reaction mixture was diluted with EA and sequentially washed with water and brine. The org. layer was dried over MgSO₄, concentrated under reduced pressure and purified by CC (Hept/EA 1:1 to 0:1) affording 170 mg (74% yield) of a colourless oil.

MS1 (ESI, m/z): 269.2 [M+H⁺]; t_{R}=0.78 min.

### K.vi. 3-(3-oxo-5-phenyl-1,3-dihydroisobenzofuran-1-yl)propanal

A solution of intermediate K.v (160 mg) in DMSO (0.37 mL) and DCM (3 mL) was sequentially treated with DIPEA (0.31 mL) and a solution of SO₃.pyridine complex (228 mg) in DMSO (0.8 mL). The reaction was further stirred at rt for 1 h. The reaction mixture was quenched with 10% aq. citric acid solution (4 mL) and diluted with DCM. The aq. layer was extracted with DCM. The combined org. layers were dried over MgSO₄, concentrated under reduced pressure affording 140 mg (88% yield) of a light brown oil.

MS1 (ESI, m/z): 307.9 [M+MeCN+H⁺]; t_{R}=0.80 min.

### Preparation L: rac-3-oxo-5-phenyl-1,3-dihydro-isobenzofuran-1-carboxylic acid

A solution of DIPEA (1.6 mL) in THF (40 mL) was cooled to −20° C., treated with n-hexyllithium (4.6 ml; 33 wt. % solution in n-hexane) and further stirred at this temperature for 10 minutes. The reaction mixture was cooled to −78° C. and treated dropwise with a suspension of 6-phenyl-1(3H)-isobenzofuranone (2.00 g; CAS 31428-41-8; prepared according to DE1941861) in THF (20 ml). The reaction mixture was further stirred at −78° C. for 20 minutes, CO₂ was bubbled through the reaction mixture for 90 minutes while the temperature slowly reached −60° C. The reaction mixture was quenched with 10% aq. citric acid solution (20 mL) and diluted with EA. The org. layer was dried over MgSO₄, concentrated under reduced pressure affording, after trituration in ether, 1.90 g (78% yield) of a light yellow solid.

MS1 (ESI, m/z): 296.1 [M+MeCN+H⁺]; t_{R}=0.74 min.

### Preparation M: rac-5-bromo-3-oxo-1,3-dihydro-isobenzofuran-1-carboxylic acid

Starting from 1,3-dihydro-3-oxo-1-isobenzofurancarboxylic acid (2.35 g; commercial; CAS 708-14-5) and NBS (3.52 g), and proceeding in analogy to preparation A, the title compound was obtained as a beige solid (1.20 g; 35% yield).

¹H NMR (DMSO d-6) δ: 8.08 (d, J=1.7 Hz, 1H), 8.00 (dd, J₁=8.2 Hz, J₂=1.8 Hz, 1H), 7.67 (d, J=8.2 Hz, 1H), 6.19 (s, 1H)

MS1 t_{R}=0.58 min (no ionisation).

### Preparation N: rac-(5-cyclohex-1-enyl-3-oxo-1,3-dihydro-isobenzofuran-1-yl)-acetic acid

Starting from compound of preparation A (150 mg) and 1-cyclohexenylboronic acid (105 mg; commercial; CAS 89490-05-1) and proceeding in analogy to preparation B, the title compound was obtained as a beige solid (37 mg; 25% yield).

MS1 (ESI, m/z): 314.1 [M+MeCN+H⁺]; t_{R}=0.80 min.

### Preparation O: rac-5-cyclohex-1-enyl-3-oxo-1,3-dihydro-isobenzofuran-1-carboxylic acid

### O.i. 6-(cyclohex-1-en-1-yl)isobenzofuran-1(3H)-one

Starting from 6-bromo-1(3H)-isobenzofuranone (673 mg; prepared according to WO2011/044506; CAS 19477-73-7) and 1-cyclohexenylboronic acid (378 mg; commercial; CAS 89490-05-1) and proceeding in analogy to preparation B, the title compound was obtained as a dark brown solid (0.74 g; 100% yield).

MS1 (ESI, m/z): 215.3 [M+H⁺]; t_{R}=0.90 min.

### O.ii. rac-5-cyclohex-1-enyl-3-oxo-1,3-dihydro-isobenzofuran-1-carboxylic acid

Starting from intermediate O.i (643 mg) and proceeding in analogy to preparation L, but using n-BuLi in hexane instead of n-hexyllithium, the title compound was obtained as a yellow solid (176 mg; 23% yield).

MS1 (ESI, m/z): 259.4 [M+H⁺]; t_{R}=0.80 min.

### Preparation P: rac-8-bromo-5-hydroxy-2,3,4,5-tetrahydro-benzo[c]azepin-1-one

A solution of intermediate F.i (2.20 g) in THF (56 mL) was treated with PPh₃ (2.25 g) and water (1.4 mL) and further stirred at 60° C. for 3.5 h. The reaction mixture was evaporated under reduced pressure and the residue was purified by CC (EA/DCM 9:1), affording 720 mg (37%) of a light yellow foam.

MS1 (ESI, m/z): 297.0 [M+H⁺]; t_{R}=0.0.56 min.

### Preparation Q: 1-(2-amino-ethyl)-7-fluoro-1H-quinolin-2-one hydrochloride

### Q.i. tert-butyl (2-(7-fluoro-2-oxoquinolin-1(2H)-yl)ethyl)carbamate

A suspension of 7-fluoro-2-oxo-1(2H)-quinolinepropanoic acid (5.00 g; CAS 1065677-04-4; prepared according to WO 2008/116815) in tBuOH (118 mL) was treated with TEA (3.6 mL) and DPPA (5.5 mL) and further stirred at 80° C. overnight. The reaction mixture was evaporated under reduced pressure and the residue was taken up in EA and extracted with water. The org layer was washed with brine, dried over MgSO4, filtered and evaporated under reduced pressure. The residue was stirred in EA and filtered. The filtrate was evaporated under reduced pressure and purified by CC (Hept/EA 1:1 to 0:1), affording 2.60 g of a mixture of product and starting material (2:1).

MS1 (ESI, m/z): 307.1 [M+H⁺]; t_{R}=0.80 min.

### Q.ii. 1-(2-amino-ethyl)-7-fluoro-1H-quinolin-2-one hydrochloride

Starting from intermediate Q.i. (2.50 g) and proceeding in analogy to preparation G, step G.ii, the title compound was obtained as a colourless solid (1.65 g; 83% yield).

MS1 (ESI, m/z): 207.2 [M+H⁺]; t_{R}=0.0.45 min.

### Preparation R: 1-(3-aminopropyl)-7-fluoroquinolin-2(1H)-one

### R.i. 7-fluoro-1-(3-hydroxypropyl)quinolin-2(1H)-one

Starting from 7-fluoro-2-oxo-1(2H)-quinolinepropanoic acid (4.00 g; CAS 1065677-04-4; prepared according to WO 2008/116815) and proceeding in analogy to preparation D, step D.i, the title compound was obtained as a beige solid (4.0 g; 100% yield).

MS1 (ESI, m/z): 222.2 [M+H⁺]; t_{R}=0.62 min.

### R.ii. methanesulfonic acid 3-(7-fluoro-2-oxo-2H-quinolin-1-yl)-propyl ester

Starting from intermediate R.i. (4.80 g) and proceeding in analogy to preparation C, step C.ii, the title compound was obtained as a yellow oil (5.0 g; 77% yield).

MS1 (ESI, m/z): 300.0 [M+H⁺]; t_{R}=0.72 min.

### R.iii. 1-(3-azido-propyl)-7-fluoro-1H-quinolin-2-one

Starting from intermediate R.ii. (4.90 g) and proceeding in analogy to preparation F, step F.i, the title compound was obtained as a yellow oil (3.65 g; 91% yield).

MS1 (ESI, m/z): 247.2 [M+H⁺]; t_{R}=0.80 min.

### R.iv. 1-(3-aminopropyl)-7-fluoroquinolin-2(1H)-one

A solution of intermediate R.iii (3.60 g) in THF (60 ml) was hydrogenated over 10% Pd/C (1.55 g) at rt overnight. The reaction mixture was filtered over a pad of Celite and the filtrate was evaporated under reduced pressure. The residue was taken up in water, the pH of the solution adjusted to pH 1 with 1N HCl and the solution was extracted with EA. The pH of the aq solution was adjusted to pH 9 with 1N NaOH and the solution was extracted with DCM. The org layer was dried over MgSO₄, filtered, evaporated under reduced pressure and purified by CC (DCM/MeOH 19:1 to 9:1+0.5% NH₄OH), affording 1.08 g (33% yield) of a light yellow solid.

MS1 (ESI, m/z): 221.2 [M+H⁺]; t_{R}=0.48 min.

### Preparation S: (R)-1-((2-aminoethyl)amino)-9-fluoro-1H-pyrrolo[3,2,1-ij]quinolin-4(2H)-one hydrochloride

### S.i. [2-((R)-9-Fluoro-4-oxo-1,2-dihydro-4H-pyrrolo[3,2,1-ij]quinolin-1-ylamino)-ethyl]-carbamic acid tert-butyl ester

A solution of (1R)-1-amino-9-fluoro-1,2-dihydro-4H-pyrrolo[3,2,1-ij]quinolin-4-one (2.75 g; CAS 1220980-47-1; prepared according to WO 2010/041194) and N-(2-oxoethyl)-carbamic acid tBu ester (2.39 g; commercial; CAS 89711-08-0) in DCM/MeOH (4:1; 50 mL) was treated with NaHB(OAc)₃ (8.58 g) and further stirred at rt overnight. The reaction mixture was diluted with DCM and water. The aq. layer was extracted with DCM. The combined org. layers were washed with water and brine, dried over MgSO₄, filtered and evaporated under reduced pressure affording 4.20 g (90% yield) of a colourless foam.

MS1 (ESI, m/z): 348.2 [M+H⁺]; t_{R}=0.55 min.

### S.ii. (R)-1-((2-aminoethyl)amino)-9-fluoro-1H-pyrrolo[3,2,1-ij]quinolin-4(2H)-one hydrochloride

Starting from intermediate S.i. (4.20 g) and proceeding in analogy to preparation G, step G.ii, the title compound was obtained as a yellowish oil (2.00 g; 67% yield).

MS1 (ESI, m/z): 248.3 [M+H⁺]; t_{R}=0.35 min.

### Preparation T: rac-2-(5-bromo-3-oxo-1,3-dihydro-isobenzofuran-1-yl)-N-[2-(7-fluoro-2-oxo-2H-quinolin-1-yl)-ethyl]-acetamide

A suspension of compound of preparation Q (121 mg) and compound of preparation A (136 mg) in DMF (5 mL) was treated with DIPEA (0.25 mL), cooled to 2° C., treated dropwise with T3P (50% solution in EtOAc, 0.3 mL) and further stirred at rt overnight. The reaction mixture was diluted with water and EA. The org. layer was dried over MgSO₄, filtered and evaporated under reduced pressure. The residue was stirred in MeOH and filtered, affording 33 mg (28% yield) of a beige solid.

MS1 (ESI, m/z): 459.0 [M+H⁺]; t_{R}=0.80 min.

### Preparation U: rac-5-iodo-3-oxo-1,3-dihydro-isobenzofuran-1-carboxylic acid [3-(7-fluoro-2-oxo-2H-quinolin-1-yl)-propyl]-amide

### U.i. rac-5-bromo-3-oxo-1,3-dihydro-isobenzofuran-1-carboxylic acid [3-(7-fluoro-2-oxo-2H-quinolin-1-yl)-propyl]-amide

Starting from intermediate R (925 mg) and intermediate M (1080 mg) and proceeding in analogy to preparation T, the title compound was obtained, after purification by CC (DCM/MeOH), as a colourless solid (1.16 g; 60% yield).

MS1 (ESI, m/z): 459.0 [M+H⁺]; t_{R}=0.84 min.

### U.ii. rac-5-iodo-3-oxo-1,3-dihydro-isobenzofuran-1-carboxylic acid [3-(7-fluoro-2-oxo-2H-quinolin-1-yl)-propyl]-amide

A suspension of intermediate U.i (1.15 g), CuI (101 mg) and NaI (764 mg) in dioxane (1.8 ml) was placed in a sealed tube, degassed with nitrogen, treated with trans-N,N′-dimethylcyclohexane diamine (0.16 mL), sealed and heated at 120° C. overnight. The reaction mixture was cooled to rt, diluted with aq. 5% NH₄OH solution and EA/MeOH (9:1). The resulting solid was collected by filtration affording 488 mg (39% yield) of a colourless solid.

MS1 (ESI, m/z): 507.0 [M+H⁺]; t_{R}=0.86 min.

### Preparation V: rac-N-[2-(5-bromo-3-oxo-1,3-dihydro-isobenzofuran-1-yl)-ethyl]-2-(7-fluoro-2-oxo-2H-quinolin-1-yl)-acetamide

Starting from 7-fluoro-2-oxo-1(2H)-quinolineacetic acid (1.26 g; commercial, CAS 1280736-31-3) and intermediate P (1.46 g) and proceeding in analogy to preparation T, the title compound was obtained as a brown solid (820 mg; 31% yield).

MS1 (ESI, m/z): 458.9 [M+H⁺]; t_{R}=0.79 min.

### Example 1

### N—((R)-9-Fluoro-4-oxo-1,2-dihydro-4H-pyrrolo[3,2,1-ij]quinolin-1-ylmethyl)-2-((R)-3-oxo-5-phenyl-1,3-dihydro-isobenzofuran-1-yl)-acetamide and N—((R)-9-Fluoro-4-oxo-1,2-dihydro-4H-pyrrolo[3,2,1-ij]quinolin-1-ylmethyl)-2-((S)-3-oxo-5-phenyl-1,3-dihydro-isobenzofuran-1-yl)-acetamide (mixture of diastereoisomers)

Starting from (R)-1-(aminomethyl)-9-fluoro-1,2-dihydro-4H-pyrrolo[3,2,1-ij]quinolin-4-one (109 mg; prepared according to WO2010/041194, CAS 1220978-22-2) and intermediate B (134 mg) and proceeding in analogy to preparation T, the title compound was obtained as a yellow solid (140 mg; 60% yield).

MS1 (ESI, m/z): 469.3 [M+H⁺]; t_{R}=0.83 min

### Example 2

### rac-6-Methoxy-4-{2-[2-(3-oxo-5-phenyl-1,3-dihydro-isobenzofuran-1-yl)-ethylamino]-ethyl}-4H-pyrido[2,3-b]pyrazin-3-one

A solution of 4-(2-aminoethyl)-6-methoxy-pyrido[2,3-b]pyrazin-3(4H)-one (67 mg; CAS 1185182-30-2; prepared according to WO 2009/104159) in DMF (1 mL) was treated with DIPEA (0.1 mL) and intermediate C (101 mg) and the reaction mixture was further stirred at 80° C. for 20 h. The reaction mixture was diluted with water and EA/MeOH. The org. layer was washed with water and brine, dried over MgSO₄, filtered and evaporated under reduced pressure. The residue was purified by CC (EA/MeOH 19:1 to 9:1+NH₄OH), affording 3 mg (2% yield) of a yellow solid.

MS1 (ESI, m/z): 457.1 [M+H⁺]; t_{R}=0.72 min.

### Example 3

### rac-7-Fluoro-1-{2-[2-(3-oxo-5-phenyl-1,3-dihydro-isobenzofuran-1-yl)-ethylamino]-ethyl}-1H-quinolin-2-one

Starting from intermediate Q (148 mg) and intermediate C (202 mg) and proceeding in analogy to example 2, the title compound was obtained as a light yellow solid (19 mg; 7% yield).

MS1 (ESI, m/z): 443.1 [M+H⁺]; t_{R}=0.73 min.

### Example 4

### rac-N-[2-(7-Fluoro-2-oxo-2H-quinolin-1-yl)-ethyl]-2-(3-oxo-5-phenyl-1,3-dihydro-isobenzofuran-1-yl)-acetamide

Starting from intermediate T (50 mg) and phenylboronic acid (20 mg; commercial; CAS 98-80-6) and proceeding in analogy to preparation B the title compound was obtained as colourless solid (14 mg; 28% yield).

MS1 (ESI, m/z): 457.1 [M+H⁺]; t_{R}=0.86 min.

1H NMR (CDCl3) δ: 8.07 (d, J=1.1 Hz, 1H), 7.84 (dd, J1=8.0 Hz, J2=1.6 Hz, 1H), 7.66 (m, 2H), 7.47 (m, 5H), 7.00 (td, J1=8.3 Hz, J2=2.2 Hz, 1H), 6.81 (m, 1H), 6.62 (d, J=9.5 Hz, 1H), 5.94 (m, 1H), 4.47 (t, J=6.5 Hz, 2H), 3.71 (m, 2H), 2.77 (m, 2H)

### Example 5

### rac-N-[2-(7-Fluoro-2-oxo-2H-quinolin-1-yl)-ethyl]-2-[5-(3-fluoro-phenyl)-3-oxo-1,3-dihydro-isobenzofuran-1-yl]-acetamide

Starting from intermediate T (150 mg) and 3-fluorophenylboronic acid (68 mg; commercial; CAS 768-35-4) and proceeding in analogy to preparation B the title compound was obtained as colourless solid (55 mg; 35% yield).

MS1 (ESI, m/z): 475.3 [M+H⁺]; t_{R}=0.88 min.

### Example 6

### rac-N-[2-(7-Fluoro-2-oxo-2H-quinolin-1-yl)-ethyl]-2-[5-(2-fluoro-phenyl)-3-oxo-1,3-dihydro-isobenzofuran-1-yl]acetamide

Starting from intermediate T (150 mg) and 2-fluorophenylboronic acid (68 mg; commercial; CAS 1993-03-9) and proceeding in analogy to preparation B the title compound was obtained as colourless solid (63 mg; 41% yield).

MS1 (ESI, m/z): 475.3 [M+H⁺]; t_{R}=0.87 min.

### Example 7

### (S)-9-Fluoro-1-[3-((R)-3-oxo-5-phenyl-1,3-dihydro-isobenzofuran-1-yl)-propylamino]-1,2-dihydro-pyrrolo[3,2,1-ij]quinolin-4-one and (S)-9-Fluoro-1-[3-((S)-3-oxo-5-phenyl-1,3-dihydro-isobenzofuran-1-yl)-propylamino]-1,2-dihydro-pyrrolo[3,2,1-ij]quinolin-4-one (mixture of diastereoisomers)

Starting from (1S)-1-amino-9-fluoro-1,2-dihydro-4H-pyrrolo[3,2,1-ij]quinolin-4-one (100 mg; CAS 1220980-46-0; prepared according to WO2010/041194) and intermediate K (130 mg;) and proceeding in analogy to preparation S, step S.i, the title compound was obtained as a light yellow foam (112 mg; 50% yield).

MS1 (ESI, m/z): 455.1 [M+H⁺]; t_{R}=0.69 min.

1H NMR (CDCl3) δ: 8.08 (m, 1H), 7.89 (ddd, J1=8.0 Hz, J2=2.7 Hz, J3=1.8 Hz, 1H), 7.67 (dd, J1=9.5 Hz, J2=3.0 Hz, 1H), 7.60 (m, 2H), 7.44 (m, 5H), 6.90 (td, J1=8.8 Hz, J2=2.7 Hz, 1H), 6.61 (dd, J1=9.5 Hz, J2=2.1 Hz, 1H), 5.55 (m, 1H), 4.97 (dd, J1=8.3 Hz, J2=3.5 Hz, 1H), 4.49 (m, 1H), 4.27 (m, 1H), 2.76 (m, 2H), 2.22 (m, 1H), 1.76 (m, 3H)

### Example 8

### rac-2-[5-(2,3-Difluoro-phenyl)-3-oxo-1,3-dihydro-isobenzofuran-1-yl]-N-[2-(7-fluoro-2-oxo-2H-quinolin-1-yl)-ethyl]-acetamide

Starting from intermediate T (150 mg) and 2,3-difluorophenylboronic acid (77 mg; commercial; CAS 121219-16-7) and proceeding in analogy to preparation B the title compound was obtained as beige solid (100 mg; 62% yield).

MS1 (ESI, m/z): 493.2 [M+H⁺]; t_{R}=0.88 min.

### Example 9

### rac-2-[5-(2,5-Difluoro-phenyl)-3-oxo-1,3-dihydro-isobenzofuran-1-yl]-N-[2-(7-fluoro-2-oxo-2H-quinolin-1-yl)-ethyl]-acetamide

Starting from intermediate T (150 mg) and 2,5-difluorophenylboronic acid (77 mg; commercial; CAS 193353-34-3) and proceeding in analogy to preparation B the title compound was obtained as beige solid (110 mg; 68% yield).

MS1 (ESI, m/z): 493.2 [M+H⁺]; t_{R}=0.88 min.

### Example 10

### rac-2-[5-(3,4-Difluoro-phenyl)-3-oxo-1,3-dihydro-isobenzofuran-1-yl]-N-[2-(7-fluoro-2-oxo-2H-quinolin-1-yl)-ethyl]-acetamide

Starting from intermediate T (150 mg) and 3,4-difluorophenylboronic acid (77 mg; commercial; CAS 168267-41-2) and proceeding in analogy to preparation B the title compound was obtained as beige solid (20 mg; 12% yield).

MS1 (ESI, m/z): 493.3 [M+H⁺]; t_{R}=0.89 min.

### Example 11

### rac-2-[5-(3,5-Difluoro-phenyl)-3-oxo-1,3-dihydro-isobenzofuran-1-yl]-N-[2-(7-fluoro-2-oxo-2H-quinolin-1-yl)-ethyl]-acetamide

Starting from intermediate T (150 mg) and 3,5-difluorophenylboronic acid (77 mg; commercial; CAS 156545-07-2) and proceeding in analogy to preparation B the title compound was obtained as beige solid (36 mg; 22% yield).

MS1 (ESI, m/z): 493.3 [M+H⁺]; t_{R}=0.89 min.

### Example 12

### rac-2-(5-Cyclopent-1-enyl-3-oxo-1,3-dihydro-isobenzofuran-1-yl)-N-[2-(7-fluoro-2-oxo-2H-quinolin-1-yl)-ethyl]-acetamide

Starting from intermediate T (150 mg) and 1-cyclopentenylboronic acid (55 mg; commercial; CAS 850036-28-1) and proceeding in analogy to preparation B the title compound was obtained as beige solid (84 mg; 58% yield).

MS1 (ESI, m/z): 447.1 [M+H⁺]; t_{R}=0.88 min.

### Example 13

### rac-3-Oxo-5-phenyl-1,3-dihydro-isobenzofuran-1-carboxylic acid [3-(7-fluoro-2-oxo-2H-quinolin-1-yl)-propyl]-amide

Starting from intermediate R (87 mg) and intermediate L (100 mg) and proceeding in analogy to preparation T the title compound was obtained as beige solid (70 mg; 39% yield).

MS1 (ESI, m/z): 457.1 [M+H⁺]; t_{R}=0.90 min.

### Example 14

### (S)-3-Oxo-5-phenyl-1,3-dihydro-isobenzofuran-1-carboxylic acid [2-((R)-9-fluoro-4-oxo-1,2-dihydro-4H-pyrrolo[3,2,1-ij]quinolin-1-ylamino)-ethyl]-amide and (S)-3-Oxo-5-phenyl-1,3-dihydro-isobenzofuran-1-carboxylic acid [2-((R)-9-fluoro-4-oxo-1,2-dihydro-4H-pyrrolo[3,2,1-ij]quinolin-1-ylamino)-ethyl]-amide (mixture of diastereoisomers)

Starting from intermediate S (97 mg) and intermediate L (100 mg) and proceeding in analogy to preparation T the title compound was obtained as beige solid (60 mg; 32% yield).

MS1 (ESI, m/z): 484.3 [M+H⁺]; t_{R}=0.66 min.

### Example 15

### rac-1-(2-{2-[5-(2,4-Difluoro-phenyl)-3-oxo-1,3-dihydro-isobenzofuran-1-yl]-ethylamino}-ethyl)-7-fluoro-1H-quinolin-2-one

Starting from intermediate Q (63 mg) and intermediate E (112 mg) and proceeding in analogy to example 2, the title compound was obtained as light yellow solid (15 mg; 10% yield).

MS1 (ESI, m/z): 479.4[M+H⁺]; t_{R}=0.74 min.

### Example 16

### rac-2-(5-Cyclohex-1-enyl-3-oxo-1,3-dihydro-isobenzofuran-1-yl)-N-[2-(7-fluoro-2-oxo-2H-quinolin-1-yl)-ethyl]-acetamide

Starting from intermediate Q (28 mg) and intermediate N (37 mg) and proceeding in analogy to preparation T the title compound was obtained as colourless solid (6 mg; 10% yield).

MS1 (ESI, m/z): 461.2 [M+H⁺]; t_{R}=0.91 min.

### Example 17

### rac-N-[2-(7-Fluoro-2-oxo-2H-quinolin-1-yl)-ethyl]-2-[5-(2-methyl-propenyl)-3-oxo-1,3-dihydro-isobenzofuran-1-yl]-acetamide

Starting from compound of preparation T (130 mg) and 2,2-dimethylethenylboronic acid (42 mg; commercial; CAS 14559-88-7) and proceeding in analogy to preparation B, the title compound was obtained as a colourless solid (3 mg; 2% yield).

MS1 (ESI, m/z): 435.0 [M+H⁺]; t_{R}=0.87 min.

### Example 18

### rac-2-(7-Fluoro-2-oxo-2H-quinolin-1-yl)-N-[2-(3-oxo-5-phenyl-1,3-dihydro-isobenzofuran-1-yl)-ethyl]-acetamide

Starting from intermediate V (130 mg) and phenylboronic acid (52 mg; commercial; CAS 98-80-6) and proceeding in analogy to preparation B the title compound was obtained as beige solid (40 mg; 31% yield).

MS1 (ESI, m/z): 457.1 [M+H⁺]; t_{R}=0.85 min.

1H NMR (DMSO d-6) δ: 8.40 (m, 1H), 8.08 (dd, J1=8.0 Hz, J2=1.7 Hz, 1H), 8.03 (d, J=1.0 Hz, 1H), 7.93 (d, J=9.5 Hz, 1H), 7.76 (m, 4H), 7.50 (m, 1H), 7.45 (m, 1H), 7.21 (dd, J1=11.8 Hz, J2=2.3 Hz, 1H), 7.10 (td, J1=8.6 Hz, J2=2.3 Hz, 1H), 6.57 (d, J=9.5 Hz, 1H), 5.70 (m, 1H), 4.87 (s, 2H), 3.28 (m, 2H), 2.24 (m, 1H), 1.86 (m, 1H)

### Example 19

### rac-N-{2-[5-(2,5-Difluoro-phenyl)-3-oxo-1,3-dihydro-isobenzofuran-1-yl]-ethyl}-2-(7-fluoro-2-oxo-2H-quinolin-1-yl)-acetamide

Starting from intermediate V (130 mg) and 2,5-difluorophenylboronic acid (67 mg; commercial; CAS 193353-34-3) and proceeding in analogy to preparation B the title compound was obtained as beige solid (34 mg; 24% yield).

MS1 (ESI, m/z): 493.2 [M+H⁺]; t_{R}=0.87 min.

### Example 20

### rac-5-(2,5-Difluoro-phenyl)-3-oxo-1,3-dihydro-isobenzofuran-1-carboxylic acid [3-(7-fluoro-2-oxo-2H-quinolin-1-yl)-propyl]-amide

Starting from intermediate U (177 mg) and 2,5-difluorophenylboronic acid (55 mg; commercial; CAS 193353-34-3) and proceeding in analogy to preparation B the title compound was obtained as colourless solid (3 mg; 2% yield).

MS1 (ESI, m/z): 494.2 [M+H⁺]; t_{R}=0.92 min.

### Example 21

### rac-5-Cyclohex-1-enyl-3-oxo-1,3-dihydro-isobenzofuran-1-carboxylic acid [3-(7-fluoro-2-oxo-2H-quinolin-1-yl)-propyl]-amide

Starting from intermediate R (145 mg) and intermediate O (170 mg) and proceeding in analogy to preparation T the title compound was obtained as colourless solid (120 mg; 39% yield).

MS1 (ESI, m/z): 461.2 [M+H⁺]; t_{R}=0.95 min.

### Example 22

### rac-N-[2-(5-Cyclohex-1-enyl-3-oxo-1,3-dihydro-isobenzofuran-1-yl)-ethyl]-2-(7-fluoro-2-oxo-2H-quinolin-1-yl)-acetamide

Starting from 7-fluoro-2-oxo-1(2H)-quinolineacetic acid (103 mg; commercial, CAS 1280736-31-3) and intermediate G (120 mg) and proceeding in analogy to preparation T, the title compound was obtained as a beige solid (70 mg; 33% yield).

MS1 (ESI, m/z): 461.3 [M+H⁺]; t_{R}=0.90 min.

### Example 23

### rac-N-[2-(5-Cyclopent-1-enyl-3-oxo-1,3-dihydro-isobenzofuran-1-yl)-ethyl]-2-(7-fluoro-2-oxo-2H-quinolin-1-yl)-acetamide

Starting from 7-fluoro-2-oxo-1(2H)-quinolineacetic acid (109 mg; commercial, CAS 1280736-31-3) and intermediate H (120 mg) and proceeding in analogy to preparation T, the title compound was obtained as a beige solid (150 mg; 68% yield).

MS1 (ESI, m/z): 447.3 [M+H⁺]; t_{R}=0.88 min.

### Example 24

### rac-N-{2-[5-(3,5-Difluoro-phenyl)-3-oxo-1,3-dihydro-isobenzofuran-1-yl]-ethyl}-2-(7-fluoro-2-oxo-2H-quinolin-1-yl)-acetamide

Starting from 7-fluoro-2-oxo-1(2H)-quinolineacetic acid (109 mg; commercial, CAS 1280736-31-3) and intermediate I (143 mg) and proceeding in analogy to preparation T, the title compound was obtained as a beige solid (100 mg; 41% yield).

MS1 (ESI, m/z): 493.2 [M+H⁺]; t_{R}=0.89 min.

### Example 25

### rac-2-(7-Fluoro-2-oxo-2H-quinolin-1-yl)-N-{2-[5-(2-methyl-propenyl)-3-oxo-1,3-dihydro-isobenzofuran-1-yl]-ethyl}-acetamide

Starting from 7-fluoro-2-oxo-1(2H)-quinolineacetic acid (103 mg; commercial, CAS 1280736-31-3) and intermediate J (125 mg) and proceeding in analogy to preparation T, the title compound was obtained as a beige solid (151 mg; 75% yield).

MS1 (ESI, m/z): 435.2 [M+H⁺]; t_{R}=0.85 min.

1H NMR (DMSO d-6) δ: 8.38 (m, 1H), 7.94 (d, J=9.6 Hz, 1H), 7.78 (m, 1H), 7.61 (s, 3H), 7.20 (m, 1H), 7.11 (m, 1H), 6.57 (d, J=9.5 Hz, 1H), 6.37 (s, 1H), 5.63 (m, 1H), 4.87 (s, 2H), 3.26 (m, 2H), 2.19 (m, 1H), 1.90 (m, 1H), 1.89 (s, 3H), 1.83 (s, 3H).

### Pharmacological Properties of the Invention Compounds

### In Vitro Assays

### Bacterial Growth Minimal Inhibitory Concentrations:

### Experimental Methods:

Minimal Inhibitory Concentrations (MICs; mg/k) were determined in cation-adjusted Mueller-Hinton Broth by a microdilution method following the description given in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA, 2006.

### Results:

All Example compounds were tested against several Gram positive and Gram negative bacteria. Typical antibacterial test results are given in Table 1 hereafter (MICs in mg/L). Staphylococcus aureus A798 is a multiply-resistant strain (in particular quinolone-resistant and methicillin-resistant), Enterococcus faecium A949 is a multiply-resistant strains (in particular quinolone-resistant and vancomycin-resistant), while Moraxella catarrhalis A894 and Streptococcus pneumonia ATTC49619 are quinolone-sensitive strains and Staphylococcus aureus ATCC29213 is a methicillin-sensitive and quinolone-sensitive strain.

TABLE 1

| | | MIC for | MIC for | MIC for | MIC for |
|---|---|---|---|---|---|
| | MIC for | S. | E. | M. | S. |
| Example | S. aureus | aureus | faecium | catarrhalis | pneumonia |
| No. | ATCC29213 | A798 | A949 | A894 | ATTC49619 |
| 1 | ≦0.016 | ≦0.016 | 0.125 | 0.25 | 0.125 |
| 2 | 0.031 | 0.125 | 0.25 | 0.5 | 0.5 |
| 3 | 0.031 | 0.125 | 0.25 | 1 | 0.125 |
| 4 | 0.125 | 0.5 | 0.5 | 8 | 1 |
| 5 | 0.063 | 0.25 | 0.25 | 1 | 0.5 |
| 6 | 0.063 | 0.25 | 0.25 | 2 | 0.5 |
| 7 | 0.031 | 0.125 | 0.125 | 2 | 0.125 |
| 8 | 0.031 | 0.25 | 0.25 | 0.5 | 0.5 |
| 9 | 0.031 | 0.125 | 0.25 | 0.125 | 0.25 |
| 10 | 0.5 | >8 | 2 | >8 | 4 |
| 11 | 0.031 | 0.25 | 0.25 | 0.125 | 0.25 |
| 12 | 0.031 | 0.25 | 0.25 | 1 | 2 |
| 13 | 0.063 | 0.125 | 0.5 | >8 | 1 |
| 14 | 0.125 | 0.125 | 0.5 | 4 | 2 |
| 15 | 0.25 | 1 | 0.5 | 4 | 0.5 |
| 16 | ≦0.016 | 0.063 | 0.25 | 0.5 | 0.5 |
| 17 | ≦0.016 | ≦0.016 | 0.063 | 0.5 | 0.5 |
| 18 | 0.25 | 2 | 0.5 | >8 | 4 |
| 19 | 0.063 | 0.125 | 0.25 | 0.25 | 0.5 |
| 20 | 0.063 | 0.063 | 1 | 0.25 | 2 |
| 21 | 0.125 | 0.125 | 4 | 2 | 4 |
| 22 | 0.031 | 0.125 | 0.5 | 1 | 1 |
| 23 | 0.25 | 1 | 0.5 | >8 | 4 |
| 24 | 0.125 | 0.5 | 0.5 | 0.5 | 2 |
| 25 | 0.063 | 0.25 | 0.5 | 4 | 1 |
| Cipro | 0.5 | >32 | >8 | ≦0.016 | 2 |

1. A compound of formula I 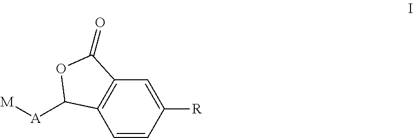
wherein
R represents phenyl optionally substituted with one or two halogen; cyclopenten-1-yl or cyclohexen-1-yl; or (C₂-C₅)alkenyl; and
the groups A and M are as follows:
A represents —NH—CH₂—CH₂—CH₂—, —NH—CH₂—CH₂—NH—CO—; or —CH₂—NH—CO—CH₂—; and
M represents 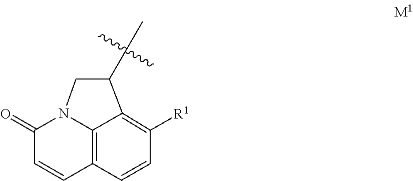
wherein R¹ represents halogen;
or A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and
M represents 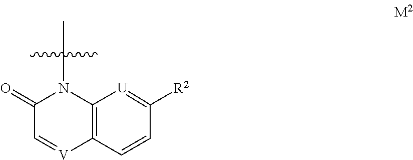
wherein R² represents (C₁-C₃)alkoxy or halogen; U represents CH or N; and V represents CH or N;
or a pharmaceutically acceptable salt thereof.

2. The compound of formula I according to claim 1, wherein
R represents phenyl; phenyl substituted with one or two fluorine substituents; cyclopenten-1-yl; cyclohexen-1-yl; 2-methylprop-1-en-1-yl; or prop-1-en-1-yl; and
the groups A and M are as follows:
A represents —NH—CH₂—CH₂—CH₂—, —NH—CH₂—CH₂—NH—CO—; or —CH₂—NH—CO—CH₂—; and
M represents 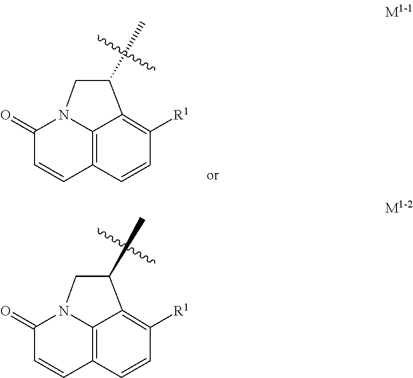
wherein R¹ represents fluorine;
or A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and
M represents 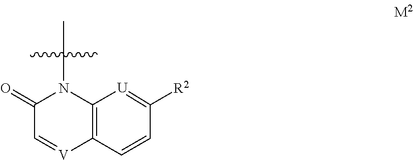
wherein
R² represents methoxy; U represents N; and V represents N; or
R² represents fluorine; U represents CH; and V represents CH;
or a pharmaceutically acceptable salt thereof.

3. The compound of formula I according to claim 1, wherein, in case M represents M²,
A represents —CH₂—CH₂—NH—CH₂—CH₂—; and R² represents methoxy; U represents N; and V represents N; or
A represents —CH₂—CH₂—NH—CO—CH₂—, —CH₂—CH₂—CH₂—NH—CO—, —CH₂—CO—NH—CH₂—CH₂— or —CH₂—CH₂—NH—CH₂—CH₂—; and R² represents fluorine; U represents CH; and V represents CH;
or a pharmaceutically acceptable salt thereof.

4. The compound of formula I according to claim 1, wherein R represents
phenyl or phenyl substituted with one or two fluorine;
cyclopenten-1-yl or cyclohexen-1-yl; or
2-methylprop-1-en-1-yl or prop-1-en-1-yl;
or a pharmaceutically acceptable salt thereof

5. The compound of formula I according to claim 1, wherein the compound is formula II 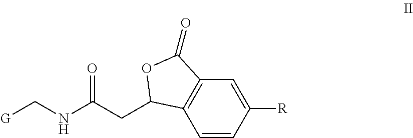
wherein
R represents phenyl optionally substituted with one or two halogen; cyclopenten-1-yl or cyclohexen-1-yl; or (C₂-C₅)alkenyl; and
G represents: 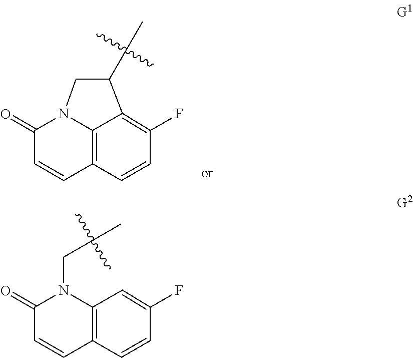 [SMILES: *C1Cn2c(=O)ccc3ccc(F)c1c32 ; *Cn1c(=O)ccc2ccc(F)cc21]
or a pharmaceutically acceptable salt thereof.

6. The compound of formula I according to claim 1, wherein the compound is:
N—((R)-9-Fluoro-4-oxo-1,2-dihydro-4H-pyrrolo[3,2,1-ij]quinolin-1-ylmethyl)-2-(3-oxo-5-phenyl-1,3-dihydro-isobenzofuran-1-yl)-acetamide;
6-Methoxy-4-{2-[2-(3-oxo-5-phenyl-1,3-dihydro-isobenzofuran-1-yl)-ethylamino]-ethyl}-4H-pyrido[2,3-b]pyrazin-3-one;
7-Fluoro-1-{2-[2-(3-oxo-5-phenyl-1,3-dihydro-isobenzofuran-1-yl)-ethylamino]-ethyl}-1H-quinolin-2-one;
N-[2-(7-Fluoro-2-oxo-2H-quinolin-1-yl)-ethyl]-2-(3-oxo-5-phenyl-1,3-dihydro-isobenzofuran-1-yl)-acetamide;
N-[2-(7-Fluoro-2-oxo-2H-quinolin-1-yl)-ethyl]-2-[5-(3-fluoro-phenyl)-3-oxo-1,3-dihydro-isobenzofuran-1-yl]-acetamide;
N-[2-(7-Fluoro-2-oxo-2H-quinolin-1-yl)-ethyl]-2-[5-(2-fluoro-phenyl)-3-oxo-1,3-dihydro-isobenzofuran-1-yl]-acetamide;
(S)-9-Fluoro-1-[3-(3-oxo-5-phenyl-1,3-dihydro-isobenzofuran-1-yl)-propylamino]-1,2-dihydro-pyrrolo[3,2,1-ij]quinolin-4-one;
2-[5-(2,3-Difluoro-phenyl)-3-oxo-1,3-dihydro-isobenzofuran-1-yl]-N-[2-(7-fluoro-2-oxo-2H-quinolin-1-yl)-ethyl]-acetamide;
2-[5-(2,5-Difluoro-phenyl)-3-oxo-1,3-dihydro-isobenzofuran-1-yl]-N-[2-(7-fluoro-2-oxo-2H-quinolin-1-yl)-ethyl]-acetamide;
2-[5-(3,4-Difluoro-phenyl)-3-oxo-1,3-dihydro-isobenzofuran-1-yl]-N-[2-(7-fluoro-2-oxo-2H-quinolin-1-yl)-ethyl]-acetamide;
2-[5-(3,5-Difluoro-phenyl)-3-oxo-1,3-dihydro-isobenzofuran-1-yl]-N-[2-(7-fluoro-2-oxo-2H-quinolin-1-yl)-ethyl]-acetamide;
2-(5-Cyclopent-1-enyl-3-oxo-1,3-dihydro-isobenzofuran-1-yl)-N-[2-(7-fluoro-2-oxo-2H-quinolin-1-yl)-ethyl]-acetamide;
3-Oxo-5-phenyl-1,3-dihydro-isobenzofuran-1-carboxylic acid [3-(7-fluoro-2-oxo-2H-quinolin-1-yl)-propyl]-amide;
3-Oxo-5-phenyl-1,3-dihydro-isobenzofuran-1-carboxylic acid [2-((R)-9-fluoro-4-oxo-1,2-dihydro-4H-pyrrolo[3,2,1-ij quinolin-1-ylamino)-ethyl]-amide;
1-(2-{2-5-(2,4-Difluoro-phenyl)-3-oxo-1,3-dihydro-isobenzofuran-1-yl]-ethylamino}-ethyl)-7-fluoro-1H-quinolin-2-one;
2-(5-Cyclohex-1-enyl-3-oxo-1,3-dihydro-isobenzofuran-1-yl)-N-[2-(7-fluoro-2-oxo-2H-quinolin-1-yl)-ethyl]-acetamide;
N-[2-(7-Fluoro-2-oxo-2H-quinolin-1-yl)-ethyl]-2-[5-(2-methyl-propenyl)-3-oxo-1,3-dihydro-isobenzofuran-1-yl]-acetamide;
2-(7-Fluoro-2-oxo-2H-quinolin-1-yl)-N-[2-(3-oxo-5-phenyl-1,3-dihydro-isobenzofuran-1-yl)-ethyl]-acetamide;
N-{2-[5-(2,5-Difluoro-phenyl)-3-oxo-1,3-dihydro-isobenzofuran-1-yl]-ethyl}-2-(7-fluoro-2-oxo-2H-quinolin-1-yl)-acetamide;
5-(2,5-Difluoro-phenyl)-3-oxo-1,3-dihydro-isobenzofuran-1-carboxylic acid [3-(7-fluoro-2-oxo-2H-quinolin-1-yl)-propyl]-amide;
5-Cyclohex-1-enyl-3-oxo-1,3-dihydro-isobenzofuran-1-carboxylic acid [3-(7-fluoro-2-oxo-2H-quinolin-1-yl)-propyl]-amide;
N-[2-(5-Cyclohex-1-enyl-3-oxo-1,3-dihydro-isobenzofuran-1-yl)-ethyl]-2-(7-fluoro-2-oxo-2H-quinolin-1-yl)-acetamide;
N-[2-(5-Cyclopent-1-enyl-3-oxo-1,3-dihydro-isobenzofuran-1-yl)-ethyl]-2-(7-fluoro-2-oxo-2H-quinolin-1-yl)-acetamide;
N-{-2-[5-(3,5-Difluoro-phenyl)-3-oxo-1,3-dihydro-isobenzofuran-1-yl]-ethyl-2-(7-fluoro-2-oxo-2H-quinolin-1-yl)-acetamide; or
2-(7-Fluoro-2-oxo-2H-quinolin-1-yl)-N-{2-[5-(2-methyl-propenyl)-3-oxo-1,3-dihydro-isobenzofuran-1-yl]-ethyl}-acetamide;
or a pharmaceutically acceptable salt thereof.

7. A composition comprising a compound of formula I according to claim 1, or a pharmaceutically acceptable salt thereof, formulated as a medicament.

8. A pharmaceutical composition comprising, as active principle, a compound of formula I according claim 1, or a pharmaceutically acceptable salt thereof, and at least one therapeutically inert excipient.

9. A method of preventing or treating a bacterial infection comprising administering to a subject in need thereof a compound of formula I according to claim 1, or a pharmaceutically acceptable salt thereof.

10. The method according to claim 9, wherein said bacterial infection is selected from respiratory tract infections, otitis media, meningitis, skin and soft tissue infections, pneumonia, sexually transmitted infections, bacteremia, endocarditis, foreign body infections, osteomyelitis, topical infections, opthalmological infections or tuberculosis.

11. The method according to claim 9, wherein said bacterial infection is mediated by Gram-positive pathogens, or Gram-negative pathogens involved in respiratory tract infections.

12. The method according to claim 9, wherein said bacterial infection is mediated by S. aureus, Enterococcus faecium, S. pneumonia, streptococci, M catarrhalis, H. influenzae, or Legionella pneumophila.

13. A method of preventing or treating a bacterial infection comprising administering to a subject in need thereof the pharmaceutical composition according to claim 8.

14. The method according to claim 13, wherein said bacterial infection is respiratory tract infections, otitis media, meningitis, skin and soft tissue infections, pneumonia, sexually transmitted infections, bacteremia, endocarditis, foreign body infections, osteomyelitis, topical infections, opthalmological infections or tuberculosis.

15. The method according to claim 13, wherein said bacterial infection is mediated by Gram-positive pathogens, or Gram-negative pathogens involved in respiratory tract infections.

16. The method according to claim 13, wherein said bacterial infection is mediated by S. aureus, Enterococcus faecium, S. pneumonia, streptococci, M catarrhalis, H. influenzae, or Legionella pneumophila.
